(19) United States
(12) Patent Application Publication
(10) Pub. No.: US 2025/0297327 A1
(43) Pub. Date: Sep. 25, 2025

(54) METHOD AND KIT FOR DETECTING CHRISTENSENELLA MINUTA

(71) Applicant: VERB BIOTICS, BOSTON, MA (US)

(72) Inventors: Georges RAWADI, PARIS (FR); Sandrine Paule CLAUS, BORDEAUX (FR); Héloïse TUDELA, CENON (FR); Guillaume DEJEAN, ARBANATS (FR)

(21) Appl. No.: 18/265,802

(22) PCT Filed: Dec. 7, 2021
(86) PCT No.: PCT/EP2021/084521

§ 371 Date: Jun. 7, 2023

(30) Foreign Application Priority Data
Dec. 8, 2020 (FR) FR2012821

(51) Int. Cl. C12Q 1/689

(52) U.S. Cl. CPC C12Q 1/689; C12Q 2600/118; C12Q 2600/16

(57) ABSTRACT

The present invention relates to an in vitro method for detecting or diagnosing the presence of at least one bacterium of the species Christensenella minuta in a subject, through the detection of at least a portion of a sequence specific for the Christensenella minuta reference genome. The present invention also relates to methods and kits for identifying, and monitoring the evolution of, C. minuta in a subject.

### TECHNICAL FIELD

The invention relates to an in vitro method for detecting or diagnosing the presence of at least one bacterium of the species Christensenella minuta in a subject, detecting at least a portion of a specific sequence of the reference genome Christensenella minuta. The present invention also relates to methods and kits for identifying, and monitoring the evolution of C. minuta in a subject.

### PRIOR ART

Our intestinal microbiota or “intestinal flora” consists of a set of non-pathogenic bacteria, viruses, parasites and fungi, or 10¹² to 10¹⁴ microorganisms present in the digestive tract of each individual, which is 2 to 10 times more than the number of cells in our body.

In 2012, a microbiologist identified and cultivated from human feces a new species: Christensenella minuta, belonging to gram-negative Clostridia. (Morotoi et al., Description of Christensenella minuta gen. nov., sp. nov., isolated from human faeces, which forms a distinct branch in the order Clostridiales, and proposal of Christensenellaceae fam. nov., International Journal of Systematic and Evolutionary Microbiology (2012), 62, 144-149).

In 2014, Goodrich et al. have identified Christensenella minuta as the most hereditary bacterial taxon in humans and also suggested its therapeutic potential.

To date, it is accepted by the scientific community that human intestinal microbiota is linked to the appearance of certain pathologies called “non-transmissible” pathologies, which defines a dysbiosis of the microbiota.

The microbial ecology has been increasingly studied in recent years as a regulator of metabolic functions and to identify its central role in establishing a healthy ecosystem, in symbiosis with their host.

Certain bacteria of the genus Christensenellaceae have thus been associated with a low body mass index in numerous human cohorts and other studies have suggested a protective role in regulating inflammation.

An absence or deficiency of the bacteria of genus Christensenellaceae is reported to be particularly involved in obesity, metabolic diseases, cardiac and vascular diseases, liver and bile duct diseases, kidney diseases, joint diseases associated with obesity, cancers and in particular cancers related to metabolism and/or dysbiosis of microbiota, autoimmune diseases, atopic dermatitis, chronic inflammatory bowel diseases, pneumonia, infectious diarrhea, food allergies, inflammatory nephrogies and neurological disorders, for example bulimia or neuropsychiatric diseases such as anxiety-related disorders or eating disorders (for example, bulimia).

The detection and identification of a bacterial seed is conventionally carried out by so-called conventional methods, such as the culturing of biological sample, the microscopic examination after staining, the blood culture, detection of bacterial antigens or detection by mass spectrometry such as MALDI-TOF. Although these methods are mostly used in routine clinical practice and allow the identification of the bacterial seed, they require a culture of at least 24 to 48 hours in pure bacterial culture, that is to say a culture comprising a single seed. However, this is only possible if the bacterium is cultivable and if the sample does not contain a large number of distinct seeds. Thus, in some cases, a faster diagnosis, independent of the culture conditions and from a more complex sample (presence of several seeds) is required.

Molecular diagnosis also allows the identification of a bacterial seed and is based on the extraction and amplification of a specific gene fragment and then its detection, in particular a 16S DNA fragment or a ribosomal DNA fragment. Molecular techniques are known to be very sensitive and specific but require quality extraction in order to reduce in particular the risks of contamination reducing the quality of the amplification. In addition, its cost is still important and they require that the fragment of interest is accessible and specific to the bacterial seed of interest. However, many species of bacteria have a very close 16S DNA which increases the presence of said bacteria in a sample, such as the bacteria of the species C. minuta.

There is therefore a clinical need for a novel method for identifying bacteria of the species C. minuta which is simple, effective, specific, fast, sensitive and economical. This is the objective of the present invention.

### SUMMARY OF THE INVENTION

To meet this goal, the invention proposes an in vitro method to detect the presence of at least one Christensenella minuta bacterium in a sample by detecting a specific sequence located between the terminals 1.921.147 bp and 2.014.152 bp of the C. minuta reference genome filed under GenBank number CP029256.1.

The term “bp” or “base pair” within the meaning of the invention refers to the pairing of two nucleic bases located on two complementary strands of DNA or RNA.

Across the entire reference genome comprising near 3 million bp, the inventors were able to identify a specific portion located between the terminals 1,921,147 bp and 2,014,152 bp. This portion is particularly suitable for identifying, detecting, and specifically diagnosing the presence of at least one bacterium of the species C. minuta. This is the purpose of the present invention.

Thus, the invention relates to an in vitro method for detecting the presence of at least one Christensenella minuta bacterium in a sample, said method comprising detecting the sequence SEQ ID NO: 1 located between the terminals 1,921,147 bp and 2,014,152 bp of the reference genome C. minuta filed under GenBank number CP029256.1. Such a portion also avoids increasing the abundance of the species C. minuta in the target sample. Preferentially, said method comprises the detection of at least 70 bp of the sequence SEQ ID NO: 1.

Advantageously, the sequence of interest to detect is located in the gene of the Bile Salt Hydrolase (BSH) located between the terminals 1,933,575 bp and 1,934,552 bp of SEQ ID NO: 2 of said reference genome.

According to another aspect, the invention relates to a method for detecting/diagnosing a disease or a subject capable of developing a disorder such as a disease and/or a dysfunction and/or an imbalance, said disorder may be, for example, intestinal dysbiosis, obesity or an inflammatory disease.

The invention also relates to a method for monitoring the evolution of the quantity of C. minuta in a subject, that is the evolution of the quantity of C. minuta in the intestinal microbiota of a subject, said method comprising:
a) implementing a method for determining and quantifying the presence of C. minuta according to the invention, in a biological sample of said subject, at a time (t0)
b) implementing a method for determining and quantifying the presence of C. minuta according to the invention, in a biological sample of said subject, at a time (t1), and
c) comparing the amount measured in a) to the amount measured in b).

The invention also relates to a method for determining or adapting a therapeutic or dietary regimen intended to prevent or combat C. minuta intestinal dysbiosis and/or C. minuta deficiency in a subject, said method comprising:
a) implementing a method for determining a level or quantity of C. minuta according to the invention, in a biological sample of said subject, and
b) comparing it with a level or quantity of C. minuta, in a biological sample of said subject after administering treatment to the subject,
c) adapting/modifying the therapeutic or dietary regimen of said subject based on the comparison of steps a) and b).

Finally, the invention relates to pairs of specific primers corresponding to the primers of SEQ ID NO: 6 and SEQ ID NO: 7 and/or primers of SEQ ID NO: 8 and SEQ ID NO: 9 and/or primers of SEQ ID NO: 10 and SEQ ID NO: 11 for targeting SEQ ID NO: 2. Preferentially, said pairs of primers make it possible to respectively target the sequences SEQ ID NO: 3 and SEQ ID NO: 4 and SEQ ID NO: 5 of the BSH gene.

The invention also relates to a kit comprising at least one pair of primers selected from the pairs of primers cited above and optionally at least one probe corresponding to SEQ ID NO: 12 or SEQ ID NO: 13 or SEQ ID NO: 15. Such a kit is able to implement one of the methods according to the invention.

Other features and advantages will emerge from the detailed description of the invention, examples and figures that follows.

### BRIEF DESCRIPTION OF THE FIGURES

FIG. 1 is a circular representation of the genome of Christensenella minuta DSM 22607 comprising the location of a preserved portion among the species Christensenella minuta.

FIG. 2A shows the results of amplification by qPCR (Syber) of the BSH gene, in particular the standard range for the pairs of primers of SEQ ID NO: 6 and SEQ ID NO: 7.

FIG. 2B shows the results of amplification by qPCR (Syber) of the BSH gene, in particular the melting curve for the pairs of primers of SEQ ID NO: 6 and SEQ ID NO: 7.

FIG. 2C shows the results of amplification by qPCR (Syber) of the BSH gene, in particular the standard range for the pairs of primers of SEQ ID NO: 8 and SEQ ID NO: 9.

FIG. 2D shows the results of amplification by qPCR (Syber) of the BSH gene, in particular the melting curve for the pairs of primers of SEQ ID NO: 8 and SEQ ID NO: 9.

FIG. 3 shows the results of amplification by qPCR (Syber) of the bacterium C. minuta 1 in CFU/g of fecal matter in samples enriched or not with a quantity of C. minuta 1 of reference for the primer pairs of SEQ ID NO: 6 and SEQ ID NO: 7 (FIG. 3A) and for the pairs of primers of SEQ ID NO: 8 and SEQ ID NO: 9 (FIG. 3B).

### DETAILED DESCRIPTION OF THE INVENTION

### Definition

The term “subject” within the meaning of the invention is understood to mean a mammal, preferentially a human or an animal. The human subject can be healthy, that is, having no symptoms of intestinal dysbiosis or C. minuta deficiency, or may be capable of developing or suspect of suffering intestinal dysbiosis or C. minuta deficiency.

The term “biological sample” within the meaning of the invention refers to samples capable of containing bacteria, in particular bacteria of the species C. minuta, for example, samples of feces, stools, colon biopsies or colon effluents.

“Evolution of the quantity of C. minuta” within the meaning of the invention is understood to mean an increase or decrease in the quantity of C. minuta in a sample, preferentially a biological sample.

The term “primers” within the meaning of the invention refers to isolated nucleic acid molecules which can or do hybridize specifically in regions 5′ or 3′ of a complementary target sequence. In general, they have a length of about 10 to 30 nucleotides and hybridize at both ends of a region containing about 50 to 200 nucleotides, preferentially 70 nucleotides. Under suitable conditions and with appropriate reagents, these primers allow the amplification of a nucleic acid molecule comprising the nucleotide sequence flanked by the primers. As they must be used in pairs, they are often called a “primer pair” or “set of primers” (for example SEQ ID NO: 6-7; SEQ ID NO: 8-9. SEQ ID NO: 10-11).

“Probes” within the meaning of the invention means molecules capable of hybridizing specifically to a sequence of interest (for example, SEQ ID NO: 3 or SEQ ID NO: 4 or SEQ ID NO: 5). They are useful to demonstrate the presence of said sequence of interest in biological samples. These probes may comprise at least one non-natural nucleotide, for example a peptide nucleic acid (PNA), a peptide nucleic acid having a phosphate group (PHONA), a bridged nucleic acid or a locked nucleic acid (BNA or LNA), and a morpholino nucleic acid. Non-natural nucleotides also comprise chemically modified nucleic acids or nucleic acid analogs such as methylphosphonate DNA or RNA, phosphorothioate DNA or RNA, phosphoramidate DNA or RNA, and 2′-O-methyl-DNA or RNA.

### Method

The present invention therefore relates to an in vitro method for detecting the presence of at least one bacterium Christensenella minuta in a sample, said method comprising detecting at least 70 bp of the sequence SEQ ID NO: 1 located between the terminals 1,921,147 bp and 2,014,152 bp of the reference genome C. minuta filed under GenBank number CP029256.1.

The reference strain of Christensenella minuta was deposited with the Leibniz Institute DSMZ (Deutsche Sammlung von Mikroorganismen und Zellkulturen GmbH, meaning “German collection of microorganisms and of cell cultures GmbH”) as number DSM 22607 and its genome is accessible as number CP029256.1 of GenBank.

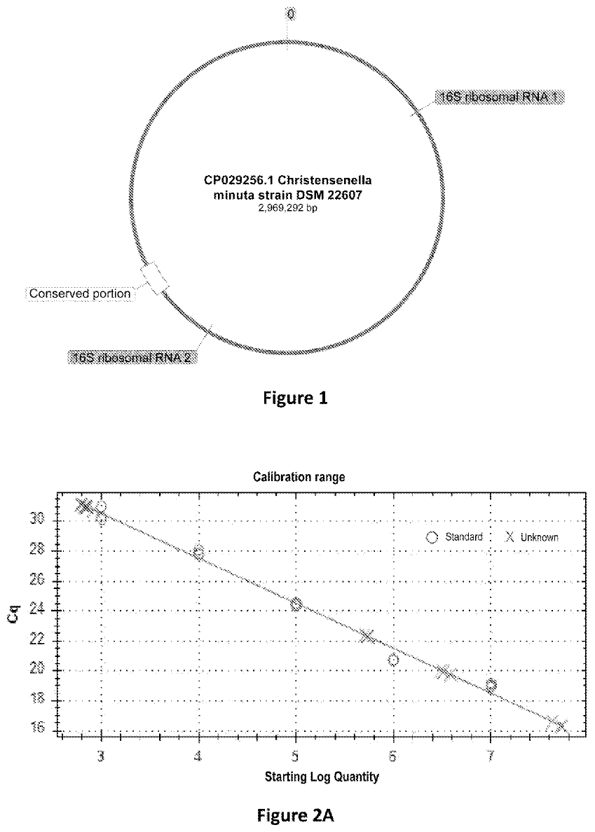

Said genome was visualized by means of SnapGene Viewer (version 5.2). The beginning of the genome was thus indicated at 0, the position reported at 0 being the position of the first database of the sequence of the FASTA file of the genome. The gene coding for the 16S ribosomal RNA 1 is located between positions 468,243 and 468,318 (locus tag B1H56_02250) and the gene coding for the 16S ribosomal RNA2 is located between positions 1,735,562 and 1,737,097 (tag locus B1H56_08210) were also indicated. The position of the preserved DNA portion is located between nucleotides 1,921,147 and 2,014,152 (see FIG. 1).

This DNA portion of the genome Christensenella minuta DSM22607 was determined, by virtue of bio-computing analyses, as particularly preserved in a certain number of strains of bacteria of this species. The results which will be presented below make it possible to demonstrate its benefit for the purposes of identification, detection, diagnosis and quantification of this bacterium in complex samples, using techniques such as PCR amplification of a fragment of this portion.

A particularly suitable sequence in the context of the present invention is the sequence SEQ ID NO: 2. It is located between the terminals 1,933,575 bp and 1,934,552 bp corresponding to the BSH gene and is present in the conserved sequence of SEQ ID NO: 1. According to one embodiment, the method according to the invention therefore comprises the detection of at least 70 bp of the sequence SEQ ID NO: 2. According to one variant, it may involve the detection of the whole sequence SEQ ID NO: 2.

The BSH gene encodes a protein involved in the deconjugation of bile acids. This gene is only present in certain prokaryotic bacteria. The BSH gene of Christensenella minuta is unique both by its nucleotide sequence and by the amino acid sequence for which it encodes. It is located between the terminals 1,933,575 b and 1,934,552 bp of the reference genome C. minuta filed under GenBank number CP029256.1. This gene's GenBank identifier is number AYH40638.1. The sequence of SEQ ID NO: 2 gives very good amplification performance for the BSH gene present in all of the bacteria belonging to the species Christensenella minuta.

TABLE 1

| | |
|---|---|
| SEQ ID NO: 2 | ATGTGTACAGCAATAACGTATTATACAAAGGATCACTATTT |
| | TGGAAGAAATCTGGATCTGGAATTTTCTTATAATGAAACAG |
| | TAACGGTTACACCAAAATATTATCCGTTTCATTTTCGTAATG |
| | GAAAAGTGCTCAATCATCATTATGCAATGATAGGGATGGC |
| | TTATATAGTTGATGACTTTCCGCTTTACTACGATGCAACAA |
| | ATGAAAAGGGTCTCAGTATGGCAGGACTGAACTTCCCGGA |
| | TAATGCGGACTATAAAGAAGTGAAGGAAGGATACGATAAT |
| | ATTGCTCCCTTTGAATTTATTCCATGGATACTCGGTCAATG |
| | TGCAAGTGTGAGTGAAGCAAGAATTCTCTTAGAACAGATTA |
| | ACTTGGTAAATTTGAACTTTAGTGAAGAGCTGCCGCTTTCT |
| | CCATTACATTGGATGATTTCTGACCAGCGAGATTCCATTGT |
| | AGTAGAATCAACAAAAGATGGTTTGAAAGTTTTCGAAAACC |
| | CTGTAGGAGTTCTGACAAATAATCCTACCTTTGACTATCAG |
| | ATGTTTAATCTCAATAATTATATGCATTTATCGAAAGAACCC |
| | CCTGCAAATACATTTGCTGCCGAATTGGAACTTGAACAGTA |
| | TAGCCGGGGTATGGGGGCGATAGGCCTTCCCGGAGATTT |
| | ATCATCCGCTTCGAGATTCGTGAAAGCAGCGTTTACAAAG |
| | ATGAACTCGGTTTCCGGTGATTCGGAATCGGAAAGTATCA |
| | GCCAGTTTTTTCACATTCTTGGCTCGGTAGAGCAGCAACG |
| | AGGCTGCGTACATCTTGGTGAAGATAAATATGAAATAACAA |
| | TTTATTCTTCCTGCTGTAACATGGACAAGGGAATTTATTAC |
| | TATACAACTTATGAAAACAACCAGATCACAGCAGTTGATAT |
| | GTATAAAGAAAATCTGGACGGAAACACTATAATAAGCTATC |
| | CGCTTATGAAAGAACAGCAGATTAACTATCGGAATTACTAA |

Particularly preferably, a specific sequence of interest of C. minuta is SEQ ID NO: 3 and/or SEQ ID NO: 4 and/or SEQ ID NO: 5. According to the invention, these sequences are in fact particularly suitable for identifying a bacterium belonging to the species C. minuta.

Thus, according to a particularly preferred embodiment, the method according to the invention aims to detect the presence in a sample of at least one Christensenella minuta bacterium, said method preferentially comprising the detection of the sequence SEQ ID NO: 3 and/or SEQ ID NO: 4 and/or SEQ ID NO: 5.

Table 2 below describes said sequences of interest.

TABLE 2

| | |
|---|---|
| SEQ ID NO: 3 | CTCGGTCAATGTGCAAGTGTGAGTGAAGCAAGAATTCTCT |
| | TAGAACAGATTAACTTGGTAAATTTGAACTTTAGTGAAGAG |
| | CTGCCGCTTTCTCCATTACATTGGATGATTTCTGACCAGCG |
| | AGATTCC |
| SEQ ID NO: 4 | AAGAACCCCCTGCAAATACATTTGCTGCCGAATTGGAACTT |
| | GAACAGTATAGCCGGGGTATGGGGGCGATAGGCCTTCCC |
| | GGAGATTTATCATCCGCTTCGAGATTCGTGAAAGCAGCGT |
| | T |
| SEQ ID NO: 5 | TGCTCCCTTTGAATTTATTCCATGGATACTCGGTCAATGTG |
| | CAAGTGTGAGTGAAGCAAGAATTCTCTTAGAACAGATTAAC |
| | TTGG |

In the context of the invention, the sample wherein the presence of C. minuta is preferentially a biological sample of a subject. According to one variant, the sample can also be a sample of soil, sediment, food, water, a medication or probiotics. A sample of soil, sediment, food or water can thus be used for purposes of looking for possible contamination. Looking for the presence of a bacterium in a medication or probiotics is also of interest for quality control purposes.

When the sample is a biological sample, the sample is preferentially selected from the feces, stools, colon biopsies and effluents from the colon of a subject.

In the context of the invention, the subject may be a human or a mammal, preferentially an animal. Thus, the method according to the invention is of interest for human and/or veterinary medicine.

The method therefore aims to detect a specific sequence belonging to a C. minuta bacterium and thus identify the presence of such a bacterium in a given sample, whether for diagnostic purposes, to verify the contamination of a sample such as an agri-food product or for quality control purposes, for example a medication or a probiotic.

Various techniques are well known to a person skilled in the art and this will be able to determine the most suitable technique for detecting the sequence of interest according to the invention in a given sample.

Preferentially, the detection of the sequence of interest is determined by a molecular amplification method such as PCR and its derivatives and/or a sequencing method such as NGS.

When it is a molecular amplification method, it may be a PCR (Polymerase Chain Reaction) or derivatives thereof.

The term “PCR derivative” within the meaning of the invention is understood to mean a variant of conventional PCR. It may thus be multiplex PCR, meta-PCR, nested PCR, asymmetric PCR, quantitative PCR (qPCR) or digital PCR in microcompartments (dPCR).

According to one variant, the detection of the sequence of interest can also be determined by sequencing such as Next Generation Sequencing or NGS.

Preferentially, the detection of the sequence of interest is determined by PCR or qPCR or dPCR.

According to a particularly preferred object, the detection of the sequence is determined by PCR using the primers of SEQ ID NO: 6 and SEQ ID NO: 7 and/or primers of SEQ ID NO: 8 and SEQ ID NO: 9 and/or primers of SEQ ID NO: 10 and SEQ ID NO: 11.

When the primer pair is SEQ ID NO: 6 and SEQ ID NO: 7, it is able to amplify, the sequence of interest SEQ ID NO: 1 or SEQ ID NO: 2, preferentially the sequence of interest SEQ ID NO: 3.

When the primer pair is SEQ ID NO: 8 and SEQ ID NO: 9, it is able to amplify, the sequence of interest SEQ ID NO: 1 or SEQ ID NO: 2, preferentially the sequence of interest SEQ ID NO: 4.

When the primer pair is SEQ ID NO: 10 and SEQ ID NO: 11, it is able to amplify, the sequence of interest SEQ ID NO: 1 or SEQ ID NO: 2, preferentially the sequence of interest SEQ ID NO: 5.

When the detection of the sequence of interest is determined by qPCR, the method also comprises probes of SEQ ID NO: 12 or SEQ ID NO: 13 or SEQ ID NO: 14.

Thus, the detection of the sequence of interest is determined by qPCR using primer pairs of SEQ ID NO: 6 and SEQ ID NO: 7 and the probe of SEQ ID NO: 12 and/or the primer pairs of SEQ ID NO: 8 and SEQ ID NO: 9 and the probe of SEQ ID NO: 13 and/or the primer pairs of SEQ ID NO: 10 and SEQ ID NO: 11 and the probe of SEQ ID NO: 14.

Table 3 below describes the sequences of the primers and probes according to the invention.

TABLE 3

| Primer Name | 5′→3′ Sequence |
|---|---|
| SEQ ID NO: 6 | CTCGGTCAATGTGCAAGTGT |
| SEQ ID NO: 7 | GGAATCTCGCTGGTCAGAAA |
| SEQ ID NO: 12 | AGTGAAGAGCTGCCGCTTTCTCCATT |
| SEQ ID NO: 8 | AAGAACCCCCTGCAAATACA |
| SEQ ID NO: 9 | AACGCTGCTTTCACGAATCT |
| SEQ ID NO: 13 | ACTGTTCAAGTTCCAATTCGGCAGCAA |
| SEQ ID NO: 10 | TGCTCCCTTTGAATTTATTCC |
| SEQ ID NO: 11 | CCAAGTTAATCTGTTCTAAGAGA |
| SEQ ID NO: 14 | CACACTTGCACATTGACCGAGTATCCA |

According to one variant, the detection and quantification of the bacterium C. minuta can also be carried out by an antibody or an aptamer specifically recognizing a portion present in the sequence SEQ ID NO: 1 and/or SEQ ID NO: 2 and/or SEQ ID NO: 3 or SEQ ID NO: 4 or SEQ ID NO: 5.

According to another subject matter, the method of the invention comprises a step of quantifying the C. minuta bacteria detected in the sample. Quantification of the C. minuta bacteria present in a sample can make it possible to use the method according to the invention for the purposes of diagnosis, patient monitoring, treatment monitoring, or adapting a therapeutic and/or dietary regimen.

When the method is used for diagnostic purposes, it makes it possible to identify a subject suffering from a disease or an imbalance or a subject likely to develop a disease or an imbalance.

Thus, the invention also relates to a method according to the invention for detecting/determining, preferentially a C. minuta intestinal dysbiosis, and/or a C. minuta deficiency in a subject. Said subject can be healthy or diseased.

Preferentially, the C. minuta intestinal dysbiosis and/or C. minuta deficiency is detected/determined in a subject, when the quantity of C. minuta bacterium detected is less than 1%, preferentially 0.4%, of the microbiota of said subject. The percentage is understood as number of bacteria relative to the total number of bacteria detected. 1% is equivalent to 10⁹ CFU of C. minuta per gram of faeces, the total number of bacteria allowed being in fact 10¹¹ CFU/g of faeces. Also, one embodiment of the invention is a method wherein the quantity of bacterium C. minuta detected is less than 10⁹ CFU/g of faeces of said subject.

The method according to the invention is preferentially able to detect/determine a disease or a subject likely to develop a disease, said disease being preferably chosen from:
Metabolic diseases, chosen from non-insulin-dependent diabetes, gestational diabetes, NASH, hepatic steatosis, pancreatic steatosis, hyperlidemia, hypercholesterolemia, infertility linked to excess weight, urinary incontinence linked to excess weight,
Other chronic metabolic diseases, including thyroiditis,
cardiac and vascular diseases, selected from atherosclerosis, thrombopathies, acute pericarditis and chronic constrictive pericarditis, arterial hypertension, vasculitis
liver and bile duct diseases, selected from hepatitis, primary biliary cirrhosis, primary sclerosing cholangitis, cirrhosis, hepatic encephalopathy, vesicular lithiasis
joint diseases linked to excess weight, chosen from osteopenia, osteoporosis, osteoarthritis, vertebral disc inflammation
neurodegenerative diseases selected from Alzheimer's disease, Parkinson's disease, motor neuron diseases such as amyotrophic lateral sclerosis, primitive lateral sclerosis and Kennedy disease
cancers linked to metabolism and/or to dysbiosis of the microbiota, selected from hepatocarcinomas, gastrointestinal tract cancers such as esophagal, stomach and colorectal cancer, pancreatic carcinoma, neuroendocrine tumours (NETs) of the gastrointestinal-pancreatic system, hepatic tumours, gallbladder and bile duct tumours, renal tumours, glioblastoma, lymphoma, multiple myeloma, chronic myeloid leukaemia, chronic myeloproliferative diseases, lung carcinoma
auto-immune diseases, selected from insulin-dependent diabetes, rheumatoid arthritis, ankylosing spondylitis, psoriatic arthritis, systemic lupus erythematosus, polyendocrine autoimmune syndrome
atopic skin diseases, chosen from eczema, psoriasis
chronic inflammatory bowel diseases, selected from Crohn's disease, hemorrhagic ulcerative rectocolitis, diverticulitis, esophagitis, gastritis, pancreatitis, gastrointestinal ulcers, irritable bowel syndrome
respiratory function disorders, selected from asthma, cystic fibrosis, chronic obstructive pulmonary disease, interstitial lung diseases and pulmonary fibroses, sleep apnoea syndrome (OSAS)
pneumonias, selected from infectious pneumonia, influenza pneumonia and avian flu, severe acute respiratory syndrome (SRAS), Pneumocystis Pneumonia
Infectious diarrhoea, selected from Clostridium difficile infection, EHEC infection, Salmonella Gastroenteritis, Campylobacter Enteritis, Food Poisoning by enterotoxin-producing bacteria, cholera, yersinosis, shigellosis, cryptosporidiosis, listeriosis
Food allergies, chosen from celiac disease, lactose intolerance, bile salt malabsorption syndrome
Inflammatory nephrogies or others related to dysbiosis of the microbiota, selected from urethritis, chronic renal failure, urolithiasis
Other inflammatory disorders, chosen from multiple sclerosis, lymphangitis
Neurological diseases related to dysbiosis of the microbiota, selected from anorexia, bulimia, depression, bipolar syndrome, autism, schizophrenia, Tourette syndrome.

According to another object, the invention relates to a method for monitoring or tracking the evolution of the quantity of C. minuta in a subject, that is the evolution of the quantity of C. minuta in the microbiota of a subject, said method comprising:
a) implementing a method according to any of the preceding embodiments to determine and quantify the presence of C. minuta, in a biological sample of said subject, at a time (t0),
b) implementing a method according to any of the preceding embodiments to determine and quantify the presence of C. minuta, in a biological sample of said subject, at a time (t1) and
c) comparing the amount measured in a) to the amount measured in b).

The subject may suffer from a disease or an imbalance such as intestinal dysbiosis and may be at risk of developing such a disease or imbalance.

According to another object, the invention relates to a method for determining or adapting a therapeutic or dietary regimen intended to prevent or combat a disease or C. minuta intestinal dysbiosis in a subject, said disease being selected from one of the above-mentioned diseases and said method comprising:
a) implementing a method according to any preceding embodiments to determine a level or quantity of C. minuta, in a biological sample of said subject, and
b) comparing it with a level or quantity of C. minuta, in a biological sample of said subject after administering treatment to the subject,
c) adapting/modifying the therapeutic or dietary regimen of said subject based on the comparison of steps a) and b).

Preferentially, the treatment administered to the subject may be a drug treatment, a surgical procedure, or a diet.

In particular, said treatment is effective if the level or quantity of C. minuta, in a biological sample of said subject is greater or increased relative to the level or quantity of C. minuta determined before the treatment.

### Primers, Probes, and Kit

According to another aspect, the invention also relates to primers suitable for use in the present invention.

Thus, the primers, in particular the pairs of primers are able to target and specifically amplify one of the sequences selected from SEQ ID NO: 1, SEQ ID NO: 2 and SEQ ID NO: 3, preferentially the sequence SEQ ID NO: 3 of the BSH gene and/or the sequence SEQ ID NO: 4 of the BSH gene and/or the sequence SEQ ID NO: 5 of the BSH gene. Preferentially, the primer pairs consist of the primers of SEQ ID NO: 6 and SEQ ID NO: 7 and/or primers of SEQ ID NO: 8 and SEQ ID NO: 9 and/or primers of SEQ ID NO: 10 and SEQ ID NO: 11.

When the primer pair of SEQ ID NO: 6 and SEQ ID NO: 7 is used, it targets and amplifies the sequence SEQ ID NO: 3 of the BSH gene. When the primer pair of SEQ ID NO: 8 and SEQ ID NO: 9 is used, it targets and amplifies the sequence SEQ ID NO: 4 of the BSH gene. When the primer pair of SEQ ID NO: 10 and SEQ ID NO: 11 is used, it targets and amplifies the sequence SEQ ID NO: 5 of the BSH gene.

Thus, the kit comprises at least two primers specifically targeting the sequence SEQ ID NO: 3 of the BSH gene and/or the sequence SEQ ID NO: 4 and/or the sequence SEQ ID NO: 5 of the BSH gene, said kit comprising the primers of SEQ ID NO: 6 and SEQ ID NO: 7 and/or primers of SEQ ID NO: 8 and SEQ ID NO: 9 and/or primers of SEQ ID NO: 10 and SEQ ID NO: 11.

According to another object, the invention relates to a kit comprising at least one primer pair (or two primers), said primer pair(s) is selected from the primer pair of SEQ ID NO: 6 and SEQ ID NO: 7, the primer pair of SEQ ID NO: 8 and SEQ ID NO: 9, the primer pair of SEQ ID NO: 10 and SEQ ID NO: 11 and mixture thereof.

Preferentially, the kit may also comprise at least one probe specifically targeting the sequence SEQ ID NO: 3 of the BSH gene and/or the sequence SEQ ID NO: 4 of the BSH gene and/or the sequence SEQ ID NO: 5 of the BSH gene, said kit comprising at least one probe selected from the probe of SEQ ID NO: 12, SEQ ID NO: 13, SEQ ID NO: 14.

When the probe is SEQ ID NO: 12, it is associated with the primers of sequence SEQ ID NO: 6 and SEQ ID NO: 7. When the probe is SEQ ID NO: 13, it is associated with the primers of sequence SEQ ID NO: 8 and SEQ ID NO: 9. When the probe is SEQ ID NO: 13, it is associated with the primers of sequence SEQ ID NO: 10 and SEQ ID NO: 11.

Finally, said kit according to the invention is suitable for implementing one of the methods according to the invention.

The invention is now illustrated by non-limiting examples of compositions according to the invention and by results.

### EXAMPLES

### Example 1

Amplification test by qPCR (Syber) of the BSH gene with the primers according to the invention (SEQ ID NO: 6 and SEQ ID NO: 7; and SEQ ID NO: 8 and SEQ ID NO: 9). Such a test makes it possible to demonstrate the efficacy of the quantification on a sample comprising only C. minuta strains and on a sample comprising a complex mixture of different bacteria.

The standard ranges were made on genomic DNA samples extracted from mouse feces and enriched with 10⁹ Colony Forming Units (CFUs) of Christensenella minuta 1.

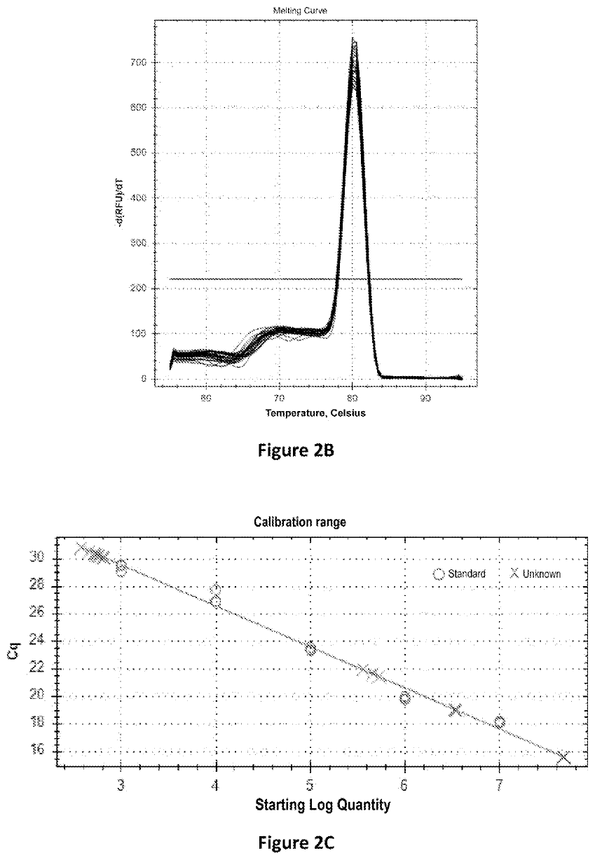

The primers were used on genomic DNA extracted from samples of mouse feces (fed with a placebo). These samples were previously artificially enriched with 109 Colony Forming Units (CFUs) Christensenella minuta 1. This matrix was then diluted in series by factors of 10 to 10 million so as to obtain a standard range for each pair of DNA primers and quantitative PCR reactions targeting our gene of interest were carried out (FIGS. 2A and 2C). These standard ranges make it possible to determine the linearity, efficiency, sensitivity and reproducibility of our test. Thus, remarkable efficiency scores (E) and confidence (R²) were obtained for the BSHqPCR2 and 3 primers on the standard ranges (respectively E=97.3%; R²=0.999 and E=98.7%; R²=0.994). The qPCR reactions were carried out with Applied Biosystems™ Power SYBR™ Green PCR Master Mix on the instrument CFX96 Touch Real-Time PCR Detection System (Bio-Rad). The thermocycler conditions were as follows: initial denaturation of 3 minutes at 98° C. followed by 40 cycles of 15 seconds at 98° C., and 15 seconds at 60° C. A melt curve was also carried out in order to verify the presence of a single amplified PCR product (FIGS. 2B and 2D). Three technical replications were carried out for each condition.

### Example 2

Amplification test by qPCR (Syber) of the BSH gene with the primers according to the invention (SEQ ID NO: 6 and SEQ ID NO: 7; and SEQ ID NO: 8 and SEQ ID NO: 9). Such a test makes it possible to demonstrate the efficacy of the quantification on a sample comprising only C. minuta strains and on a sample comprising a complex mixture of different bacteria.

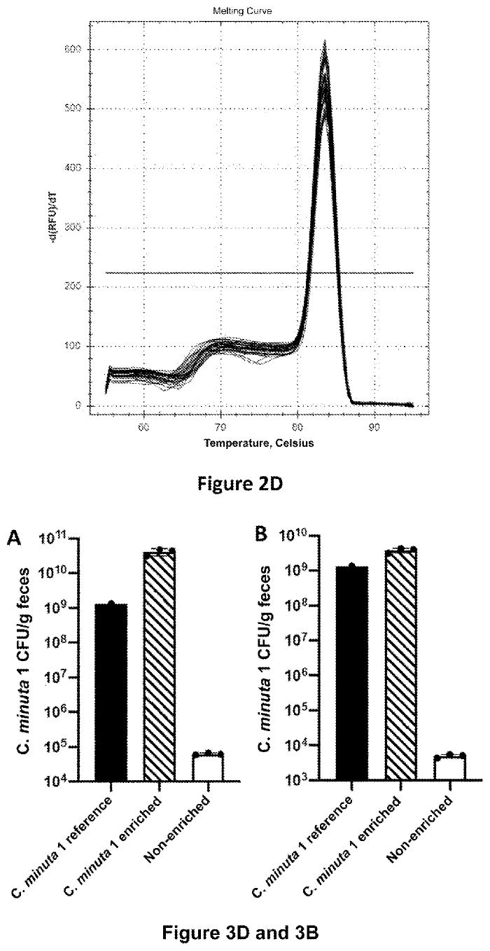

Two samples of genomic DNA extracted from mouse feces were then used to validate our method for detecting and quantifying the strain Christensenella minuta 1: a negative control not enriched with C. minuta, and a reference extract previously enriched with a determined quantity of C. minuta (FIGS. 3A and 3B). Three technical replications were carried out for each condition. The quantity of C. minuta expressed in CFU/g of fecal matter could be determined using the standard range. Thus, quantification of C. minuta 1 in the “C. minuta 1 enriched” sample robustly reflects the expected quantity, represented by the “C. minuta 1 reference” sample. The primer pairs of SEQ ID NO: 8 and SEQ ID NO: 9 demonstrate best results as to quantification of Christensenella minuta 1 within this complex matrix (FIG. 3B). Indeed, the quantization delta between the expected number CFU/g and the number found in our “C. minuta 1 enriched sample” is more robust (FIG. 3B).

1. An in vitro method for detecting the presence of at least one Christensenella minuta bacterium in a sample, said method comprising detecting at least 70 bp of the sequence SEQ ID NO: 2 located between the terminals 1,933,575 bp and 1,934,552 bp corresponding to the BSH gene of the reference genome C. minuta filed under GenBank number CP029256.1.

2. The method of claim 1, characterized in that the sample is a biological sample of a subject or a sample of soil or a medication or probiotic.

3. The method of claim 2, characterized in that the subject is a human or an animal.

4. The method of claim 3, wherein said method comprises the detection of the sequence SEQ ID NO: 3 and/or SEQ ID NO: 4 and/or SEQ ID NO: 5.

5. The method of claim 2, wherein the biological sample is a sample selected from faeces, stools, colon biopsies and colon effluents of said subject.

6. The method of claim 1, wherein the detection of the sequence is carried out by a so-called PCR molecular amplification method and/or by a sequencing method.

7. The method of claim 1, wherein the detection of the sequence is carried out by PCR using the primers of SEQ ID NO: 5 and SEQ ID NO: 6 and/or primers of SEQ ID NO: 7 and SEQ ID NO: 8 and/or primers of SEQ ID NO: 9 and SEQ ID NO: 10.

8. The method of claim 1, wherein the method comprises a step of quantifying the C. minuta bacteria detected in the sample.

9. The method of claim 2, for detecting C. minuta intestinal dysbiosis and/or a C. minuta deficiency in a subject.

10. The method of claim 9, wherein the quantity of C. minuta bacterium detected is less than 10⁹ CFU/g of faeces of said subject.

11. The method of claim 9, for detecting a disease or a subject likely to develop a disease, said disease is chosen from:
Metabolic diseases, chosen from non-insulin-dependent diabetes, gestational diabetes, NASH, hepatic steatosis, pancreatic steatosis, hyperlipemia, hypercholesterolemia, infertility linked to excess weight, urinary incontinence linked to excess weight,
Other chronic metabolic diseases, including thyroiditis,
cardiac and vascular diseases, selected from atherosclerosis, thrombopathies, acute pericarditis and chronic constrictive pericarditis, arterial hypertension, vasculitis
liver and bile duct diseases, selected from hepatitis, primary biliary cirrhosis, primary sclerosing cholangitis, cirrhosis, hepatic encephalopathy, vesicular lithiasis
joint diseases linked to excess weight, chosen from osteopenia, osteoporosis, osteoarthritis, vertebral disc inflammation
neurodegenerative diseases selected from Alzheimer's disease, Parkinson's disease, motor neuron diseases such as amyotrophic lateral sclerosis, primitive lateral sclerosis and Kennedy disease
cancers linked to metabolism and/or to dysbiosis of the microbiota, selected from hepatocarcinomas, gastrointestinal tract cancers such as oesophageal, stomach and colorectal cancer, pancreatic carcinoma, neuroendocrine tumours (NETs) of the gastrointestinal-pancreatic system, hepatic tumours, gallbladder and bile duct tumours, renal tumours, glioblastoma, lymphoma, multiple myeloma, chronic myeloid leukaemia, chronic myeloproliferative diseases, lung carcinoma
auto-immune diseases, selected from insulin-dependent diabetes, rheumatoid arthritis, ankylosing spondylitis, psoriatic arthritis, systemic lupus erythematosus, polyendocrine autoimmune syndrome
atopic skin diseases, chosen from eczema, psoriasis
chronic inflammatory bowel diseases, selected from Crohn's disease, hemorrhagic ulcerative rectocolitis, diverticulitis, esophagitis, gastritis, pancreatitis, gastrointestinal ulcers, irritable bowel syndrome
respiratory function disorders, selected from asthma, cystic fibrosis, chronic obstructive pulmonary disease, interstitial lung diseases and pulmonary fibroses, sleep apnoea syndrome (OSAS)
pneumonias, selected from infectious pneumonia, influenza pneumonia and avian flu, severe acute respiratory syndrome (SRAS), Pneumocystis pneumonia
Infectious diarrhoea, selected from Clostridium difficile infection, EHEC infection, Salmonella gastroenteritis, Campylobacter enteritis, food poisoning by enterotoxin-producing bacteria, cholera, yersinosis, shigellosis, cryptosporidiosis, listeriosis
Food allergies, chosen from celiac disease, lactose intolerance, bile salt malabsorption syndrome
Inflammatory nephrogies or others related to dysbiosis of the microbiota, selected from urethritis, chronic renal failure, urolithiasis
Other inflammatory disorders, chosen from multiple sclerosis, lymphangitis
Neurological diseases related to dysbiosis of the microbiota, selected from anorexia, bulimia, depression, bipolar syndrome, autism, schizophrenia, Tourette syndrome.

12. A method for monitoring the evolution of the quantity of C. minuta in a subject, said method comprising:
a) implementing a method according to one of the preceding claims to determine and quantify the presence of C. minuta, in a biological sample of said subject, at a time (t0)
b) implementing a method according to one of the preceding claims to determine and quantify the presence of C. minuta, in a biological sample of said subject, at a time (t1), and
c) comparing the amount measured in a) to the amount measured in b).

13. A method for determining or adapting a therapeutic or dietary regimen intended to prevent or combat C. minuta intestinal dysbiosis in a subject, said method comprising:
a) implementing a method according to one of the claims 1 to 11 to determine a level or quantity of C. minuta, in a biological sample of said subject, and
b) comparing it with a level or quantity of C. minuta, in a biological sample of said subject after administering treatment to the subject,
c) adapting/modifying the therapeutic or dietary regimen of said subject based on the comparison of steps a) and b).

14. A kit comprising at least two primers specifically targeting the sequence SEQ ID NO: 3 of the BSH gene and/or the sequence SEQ ID NO: 4 and/or the sequence SEQ ID NO: 5 of the BSH gene, said kit comprising the primers of SEQ ID NO: 6 and SEQ ID NO: 7 and/or primers of SEQ ID NO: 8 and SEQ ID NO: 9 and/or primers of SEQ ID NO: 10 and SEQ ID NO: 11.

15. The kit according to claim 14, also comprising at least one probe specifically targeting the sequence SEQ ID NO: 3 of the BSH gene or the sequence SEQ ID NO: 4 of the BSH gene or the sequence SEQ ID NO: 5 of the BSH gene, said kit comprising the probes of SEQ ID NO: 12 or SEQ ID NO: 13 or SEQ ID NO: 14.


SEQUENCE LISTING (14 sequences)

SEQ ID NO: 1 - DNA, 93006 bp, Christensenella minuta
  misc_feature: >CP029256.11921147-2014152
atttgtagtgtaaatcagcgtgcggacagtctgtgaacacttgaaatatactatgtagtgtaagtactaatagtatgttacactatgaagagttacaggagcagacaaaggatttcatgatggtactcaaacaaacgcacgagcgccccccaggtgaaggagtttgcttcacgccgagctggagtaaccatagcagtccaaaagccgggatgtacagcatcgcaaaattcacaatatggagctataaggtggatgtggtatagcgtcggaaagtccgaaaatatctgcaaagtgttctgttccggcataaaaaacgcttttcttacgaagaacgtgcggcttatatggttatttttgaattatcctgtgctttttggtagagtcgctataatagtattattattgcagattctcttgaaccgttgaggggcagacattcggcatatagtatttacagagccatattcgacatatgataagaccgcgccgccattgaattcgatatgaaagtatgttaaaaaggaggccagtatgatacagaaaaaaatattgaaaccgtttttatgtcttttatgtattctcgctattcttgcatctgccggatgtggtatgatgggggagctattgaacgttcttgaggaagaaccgaaacccacgaaagagctggaatctacggaagatttaactcagccgagcagagagccgtatactagacgatattattctagcgatgtcgagcgttttgatgtcccgcttggggaacgctccagggagtttttgacaaggatgcttttgtcatcgtattctttgaattcaaggacattatagaagcccaatacactgaagaaaacgaaacgaggcttctcgagctttatgaggaaattaggtccggcctgctggatgttaaaacggacgttagccttgcgaagtatgattattaccttgttatagatgattttttttcgatactttggtgtaaatgtaaatatgatatgatacttacatatccacttgctatgggctaaacttttatccatttgaaaaactcctttcgagtagaatgtagatgttcaggctacatgaaaacgagaaaggagttttttgccctgtaagctttgtttcccacccgtatatagaccgtaccggataaaaaggacagtttccgaaaagccacccccgctgtaggatagaactacaacgggggtgaaaatgtgtatgtcaagaaaatacacacaaatcaaaggatattatgaagaaatggaacgccagcaccaagaaggctgcagctgtcgggaaatcggagaaaagttaggctttacaaaagaacaggtgaaagaatgcctaagaagaccaagacgcaaagcccaaaaacagaacgccagtatcccaaatcggcgtggtcgaccggctaagcaaatgccttgcattctcagtgcattgtaaaaagaggtcaggcagcttcggatggaaaatgaattgctgcgggattttctgaaaacaaccgaaaggaagtgagaacgtctgccaaatatcaggtgatacaaaaatacgcatcgaagtatccaatcattcgcatgtgtgtattctttggggtatcccgcagcggatactacagctttctaaagcggaggatacagccaaaccgggaatccgcattactggggcgaattcaagaatgccgcggcagtggaagatatatgaatacctacggctgtcatcgggtacagatttggatcaaacgccagtacggtgaatactacaattacaagacggtgtggcgtgttatgcacaaatacgggctgctatctaagattcgccgaaagagattctttcattgcggtgagcagatccgtacctaccccaatctattgaatcggaacttccacgccgacaagcctaaccagaaatgggtaacagacatcagttgcatcccaaccagtcagggccccctacctttccatcattcgggatttgtttgatttaagcattgccgcttacaaaacaagtatccatcagagcattaagctggtacttgatatgatacggttggcgatggaaaaagaaacagtcaccgcagagttgcagctccacagtgatcttttctttgcgtttgacatctttgagcgtcttcaataccaagttgatcgattgctctgtgcctgtgcgatgcgctacgatacttaggtcgtaaaggcctcgtatgaccgacaggtacaccgtgccttgttctgtgggggtatatgagtgaaatgaatccggactatcgggaggcgctctatctcacctattttgaggatatgagatatgcgcaggcggcggaggtcacgggaaagacggtgaagcagattaccaacatggtatatcgcggaaaagagagcttgcgcaaactgctggaacgggaggggatcacaaatgcggagtcatgcggaacgggtcgcggataccggcgccgtcgttcccggctcgttcagcttcaagaaatatgaagacggcagagtatacccggacgaaaaccgcatggaaatcctctttacccctgatgatacggaggcatacgccactgcggaatgcactgttaccgtgaccggaataaaatacacaatcacaagcgtcccctacgacatctacatcaacgacaagctgccggggacggcgtttgagaatttagggcttccttccggcatcaacatagaggtcagcaccggcgcgaactttagttccgttcccgtgacatgggacagttcctcctacggcccggactcttatgaggagcagatcattaccggcgatctgcatgttgaaaagagttcatacgcggatcttctggagccgacaagcacagtcaaggctactgccaggataaagctcattgataaccgtgtctttacgcccacgcttgagccgcccacctactccaagcagctctacggaggcgatttgtgcttccttgtcttttctgacaactacctctccggcggaaaggccacggtgaacggcgaaacggttcccggcacgttcaagctggacaaaaatcagaaaatatacggacactcattgacggcgagccagctcctgcaggccgagccgctgcagctgaagggcatcttcactcccaacgaccagaagcggtacaccacggcggaatgtaccgtcggtgtgaatgtcattccccgcagatttgtcaaacccagttacaacagggtgcttaacaacacaaagatcggcatgacctttgaggagctgaacctgtcggagtcgttcagtttcttcgtggttagcgacgaaactccccccggagagctcgagtcgtattctatgaatgttgtcacctcgtgggacgcttcaacttatgaccccaattcgtatgatatgcagacgatctacggtgaagtcgaccgcgccgaattggagaagtatttcaccataccagaggatgccaacctcagggctgtgatcaaggttcagctgcagcccgacccagccgaaacggagatcacgcaggcacccgtattccgctggaaaaacggtgattttacgctgtccgacaacaccatcttctcccagcgaaggtttcaggtgggaacctacaacgcggaagtcggcagtctctttggcggcgtggtaaaggtcaaggggacggataccgtcgtccccggcacattctcatttaagaagggtacgccgctctactcctcgtctccgggcgaatacgacatgacggtggtgttcacgccgacagatccgcgcctccacaagccgacggagaccagcatcaaaataaacgtcatcaaaaacaccctcaaggatgccgatgagccggaacccattaccgataagccgctcggcacgccctttacggagcttgggctgccgaaagatgtggtcgtcaatgccgttgacggcgcttggcgctggattgacgtagcatgggatgcggcgtcctatgacgcggagtccccggattggcagaccatcaccggtacgttggtgtttgatgaatatgacgaaagcacctaccagcagccaggccctgtggtaacggccacgatcagggtaaagctcatcgtcccggattccgcgcctaccgtcaccaccggaaccctgccgacggcttgacgcttaacgcggatacaggcgaaatcagtggcacgcccactgcggcgggcactgcggtctttacggtcaaggtggtcaacgttttgggttcggacacaaaggagctgtcccttgcgatagtcaaggccgagcacaccgccacctttgacggcaacggtggtatgccctctgtcggcagtatgaccaccacgaatcagaagctggtctctctgcccgatgcttcccgcagcaagcacagcttcgacggctggtacaccgaaaagagcggcggcagcgacaacccgcccgtcaccgattatgcgctgcgctttgaaataggaggcggcagccatattgccgacgtgcggggaacgtacaacacctacattgacctgacaaagtacgttccgatccggcgcggctatacctttatcggctggtacagcgaccgcgatctgacaaacaaggtttccggcgtttatctgacgaacgatatgaccgtatatgcaggctggcgcgtgactgcaatcccgcagaccggtgacggcagccaactgggtttgtggagtattacgctatgtgcgtctcttgccggatgtctcgcgctgaccacatggcagctcagacggcgcaagggcgcatcacgggaaactgcagaaaagtaatcagcacacctttgtggcacgggtttgttcccccgtgccacattcgctatcttcccaggaaaaagacggctgtccttttgaagaacagccgtcctgcttttactgttgatgtgtatgcacctggatatcattcctcaaacaggtacaggagatagccgtacccatcggactccatctttgcataaggcacaaagcggagggacgcactgttgatgcagtagcgcacgccgttgggcgattccggatcaccggtaaatacatgaccgagatgagagtctccagcgcggcttctgacctccgtgcggcgcataccatggctcaggtcctccagctccaccacggcaggctcctcaatgggcttggtaaaggcgggccagccgcagccgctctcgaacttgtccgtggaggaaaagagcggttcgccggtaacgatgtccacatagatgcctttctcgaactggttccagaactcattgctgaagggacgctccgtgccgctctcctgggtgacccggtactgctccgccgtcagcttgtcccggacggactccgccgcgggcttttggtagtcgcccggatcgatgcgcagcttggagaacagctccatctccgcttttgggatatggcagtagccgttcggattcttctccagatagtcctggtggtactcctcggcgggatagtagtttttcagcgggccgatctccacgaagaacttctcgctgcgtccccgctcaatctcggcgatgcgttccaccgtctccttcgcgctttcgttagtgtagtagacgccggtctggtactggctcccgatgtcgttgccctgccggttttctaccgtagggtcggtgacgtagaagtaggccagcagcagggcgtcgagacttatctgctcggggtcgtattccacccgcacggtctcccggaagccggtgttccccttgcagacggtctgatagtcggcgtccgcctcgcaggtaccgttggcgtagccgctctcggcatccagaacgccggggatggactgcatcagctgttccagtccccaaaagcagccgcctgccagatagatgacattttccgtggtatccataaacatgctctcctcgccggacatatcagagggatgcccggccatttccttcgattccgttttctgcggcatattcttatgtgctgcggcgcagccgctcagtagcagtgccgcagcaagcagaagcggtaatactctccgatacatggtggtttcctcctttctactacaataatttatggataggatatggcagccagccatacgcctttccatcccatagtctaagctacaataaggggagtaattggtctgacgactcctttcttgggtctacacgcccgtacgtgagacagtcagccatcctcttgttgcagcgttcgttttgcaggtagagccaccactcgtgcgttcgtgtcactcagtagattgaataggcaatgggtaagagatgggcaccgattacaatacatcaatcaggaggaatacttatgaacgctgtcggtatcgacatttccaaaggcaagtgcatggttgctatcctgcgtcccttcggtgaggtggttgcttcaccttttgaaattcagcacaccgctcatgaactcagacgtttggttgaacgtctgaaaagtttggacggtgaaacccgcattgttatggagcatactggcaattactatctgcctgtcgccaggcatctgcacgaagccggcctctatgtgtccgttgtcaatgctattcttgtacatgactacggtcaaaattcccttagacgggtcaaaacggacaagatagatgctgttaaaatcgccagctatgctctaagccattggcttgatcttccgcaatatgcgccggaagaagacgttcgccacatgcttaaaacctgcgcccgccagtgcaaccagtacgttaagcagagtgttatgcttaaaaacagcctcatcgctttgcttgaccaaacctttccgagtgtcaacatgctgttctccagcccgccccgcaagggggacggtcacgaaaaatggattgacttcgtgggtaaattttggcattttgaatgcgtcaacagcctttccaaaaacgtttttacccagcgctatcaaaaatggtgcaaaagaacaggttacaactttctttccactaaggccgctgccatctacgatttcgcctgcatacaagtcagtacactgcctcgaagcacctttgccaaacagcttattactcaagctgtgtcgcagcttaactccctatcggaaacgctggctctgattcggcagcagatgctttcccttgcctccggcttgccggaatttcctgttgtcatggctatgcaaggggtcggcaatgtgttaggacctcaactcatggctgaaattggcgatgtgcgacgtttcaaccgtaagcagtcgctggttgcttttgccggtatcgacgcaccgcctttccagtccggcgcctttgaagcacagagtaccggcatttccaaacgaggttcttcctcactcagaaagacgctttttcaggtcatggactgcctgctcaaacacgcgccctctcatgatgctgtattccaatatcttgacaaaaaacgcggcgagggtaagcattattacgtctacatgatggcaggagccaacaagttcctgcgtatctactacgcacgggttaaggagcatctaggcagtttggcggttcagtagcccacatattctttttgcttggcttaccagcttatttcagggtggttttatttgatgttccattttttacctttcattttttattcatttccggcttgacttttctttgcaggtctgaggagataaattcgcttgtcagcccatatcggcggcaagagcctggtcgatgagcttttgcagcgtctccgcctgctttttttccgtgacggcacctgtgatgggctctcccacgatgttgccgttgcggtcaaccacataggtggtgggataagcaaagatgttcgtggtaaactttcccgcctcgccgtcggaagcgaaatacacattctgataagtcgcgcctttcttggcgagtacatctttcgcctcagaaattgccgctttgtcgccgtccagtgtgaaggtgttgacaccgatgagcgcgccgcccttctcagtaagctccttgttcagcgcatccaactcggcaagctcgcccacgcagggattgcaggtggtaaaccaaaaattcactacggtgacggcgttgccggagaatagctcatcgctcttcaccgcgttcccatccaagtctttaccctcaaatgcagggaacttctgcatactgccgtcatcgggcggcgtggtcatgtcgctgcccgcaggcatactcatggttccatccatggattgctgtgccgcctcggggtattttttctcaatcatggtcagcttgttttcgatgtcgcggattttctccgcttcttcctgaagcagtttcagctcatccgccgtaaactgatccttggcggattcgattgtactgagcaggaaatcaccgtagtttttgccgtcttcctgcatggtcatgcccttgtcggccgccatgaataccttttcccaaagcgctgtgttctccgagagaatggcattttcctgtgccagcagatctttgtgcatggcagccgcttcctcggcgttcttcggctcattgctcatcccatccattttgtcgccgccctttgcgccgcaggctgccagagacagcaccatcagtgcgagaagcaacagcgccagcaatcggccggcagtattcattctattcatccgttttgttcctccttgttattgttgtttttctgcggctgttctgccgcctttgttttatctttaccgtttccgaagccgtagcggaagcacacggcgttggtcggacaggctcggacgcacatcccgcagcgaatgcactctgtatggttgggcgtctttgtcacatccacatccattttgcaggctctggcgcattttccgcaggagacgcacttgtttttgtccaccttcatctggaacagggacaccctgttgaacagcgcgtagaacgcgcccagcgggcagagccacttgcagaagggacggtagaatacgatactcagcgcaatcaccgccagcaggatgctgaacttccatgtaaacaggctgcccagcgccgcccgaatgccggaattggcaagggacagcggaatggccccctccagcacgccctgcgggcagaggtatttgcagaagaaagggtcgcccatgcccacatcgttcacaaggaatgccggcagcagcaaaaccatcaccagcaaaacggcgtacttgaggtatcgcagcggcttcagccttttcgtggacagcttcttcgtgggaatcttgtgcagcagctcctgaaaccagccgaagggacacagaaaaccgcagataaagcgtcccagcaggactcccagcaaaatgaggaagcctgtgatataataggagaagctgaacttggaagaccctaccaccgcctgaaacgccccgatggggcaagcgccggaggccgccgggcaggagtagcagttcaaccccggcacacagacggttttccctgcgccctgatacagtccacccctgaggaaattcggcaggtgcaggttggtcaataaagttgcccccgcctgtatccagccccggaagcgggacatagtctgtgacacgcccgaaaacttcttacccaatgcccacacactccagacataatttgattgctttgctcagcaccgccgccgcctcgccgcgccatacgccgaagcacagcatggcgattcccgcaaccagcagcaaggcctgcgccgcagtcttttttacatgactcaattccatcaccctttcgttttttctgccaccatcataacgcgagaaagttaaaatccctgttaagaacgaaaatttaacgcaaaccttatctgtttttgctataatttaagaacaacatgaaaatgaagaataaaaaggagggctctcatgcaccttttagtaattgaagatgaacgcgccctgtgcgagaccatcgtccgcagcctgcggcggctggcctacagcgtagattactgctacgacggggagaaggcgctggagctgatcgccgttgaaaaatacgatctcatcctgctggatctgaacctgccggggaaggacggcatgacggtgctgcgcaccctgcggcagacagaccgggagacgagggtgctgatcctctccgcccgcagcgaggtggaggacaaggtggaggggctggatgcgggcgccaacgactatctggcaaagccctttcacttagccgagctggaggcccgcattcgcagcctgactctgcggcagttcacccagcaggatgtgctgttgacctgcggagccttgacctttgacacgcgctcccgcaccgcttccgtcaacgggcagacattgacgctgacccgcaaggaaacgggcattctggaatatctgatggtgcaccaggggcggcctgtgagtcaggaggagctgatggatcatgtctgggacaacagcgtggacagcttcagcaactccattcgggtacatatctccgccctgcgcaaaaagctccgggtcgccctgggttacgaccccatccgcaaccgtatcggcgagggctatctgatgggaggcgaggagacatgaggaagatatctttgaaatggcgcatcaccctgatgaccgtcctgcttatcggcatcacctgcgtcgccatgaatctgctgctctgctcctccggtgtgtactatatggacacgatcgcagacaccctgcagggcggcggcactattattatgaacggtgaaggggcagagagctttgatccgcagctcataacgcctgacgaggatctgaccatcgttgtcaatggagcacaggggcgttttcgtacgaccaactggtacatcacggctgtggtgacactgctcagcggcattctggcctattttgtcagcgggcgcgccctcaaacccctgcgcagcttcgcttctcaggtggagaaggtgcagctgaataatctggcggatatgagaaccaatgaggatgttctgccggagtttcggcagctgagtcactccttcaaccagatgctggagcggctgaacaacgcctttgccgctcaacggcagttcaccggcaacgccgcccacgagctacgcacgccgctggcgctgatgcaggcgcagctggatctgttttccgcggagcatcccgatgtgctgccggagacggcggaatttctctccctgctgcaggagcagacggagcggctggcgcagatgacaaaaacgctactggaaatgagcgaccttcaaacagtagcgcgtaccgaccaaatccagctgggaccgatgatcgaggagatcttcgcggatcttgcgccgctggcggaaaaaaacgatatcatgctggagcgaaagggcgacggcgccatgaccggcaacgacacgctgatctaccgaatgcttttcaacctgacggaaaacgccatcaagtacgaccgtcccggaggcgcagtgcgcatttccatatcccaggaggaaaagcgccttctcatccgcatcatggataccggttgcggcatcccgaaagaataccggcagagtatcttccagcccttcttccgggtagataagtcccgtagcagggaatacggcggcgcagggctggggctgtcgctagcatgggagattgccaatctccatggtggttccgtatgggtcgagaagagcacagaggaaggaaccaccattgctgtggaagtcccagcgtaataaatcaaaactccccattaaacaagtggtttgatctagccttataagagctaatcagctatggcagtttccttaaggagacatcgaatagcgcggtgaccacattttatcctttggcggcccctacccaagggcctctttgtttctatttcagattgatcgagctttcccagataatctgtcgccaactaaaacgtactgctctaaaaaggctttacacactggaagagcaagtaccgaaaacaaatcatccttatctttgaaataccgataaagggcacctgttgtaaggttagcaccggccatgatctggcgcaaggatactccggtaaaactatattctaagaattccttttggcacttttaagggtatcgccatgtgttttatcatattctgcaatcaatataacttcctccccgtaatgttgttatcttgatctttattgtcaagattttgagtatgaaatgtctcataatagtgcctaaaaatttccccacgcccttatctaaaaattacacatgcaaaagaaaatagccctttttaaatcgactctataatttgaattaacagcttgtcctcatcaaacaattcgtatagattattttggaggacaaataataaataatatagaaaaatttaatattgtattggaaaagctattgcaaaatatgtcgattattttcatttgcgtgacagtttaaataattataaagcgtttataaaatatatttagatagagcataggttgttccagaatttacaatagatctaaaaggatcgaacatgttgtagtaaaaggttattgaggaggcgatacaaatgtgtacagcaataacgtattatacaaaggatcactattttggaagaaatctggatctggaattttcttataatgaaacagtaacggttacaccaaaatattatccgtttcattttcgtaatggaaaagtgctcaatcatcattatgcaatgatagggatggcttatatagttgatgactttccgctttactacgatgcaacaaatgaaaagggtctcagtatggcaggactgaacttcccggataatgcggactataaagaagtgaaggaaggatacgataatattgctccctttgaatttattccatggatactcggtcaatgtgcaagtgtgagtgaagcaagaattctcttagaacagattaacttggtaaatttgaactttagtgaagagctgccgctttctccattacattggatgatttctgaccagcgagattccattgtagtagaatcaacaaaagatggtttgaaagttttcgaaaaccctgtaggagttctgacaaataatcctacctttgactatcagatgtttaatctcaataattatatgcatttatcgaaagaaccccctgcaaatacatttgctgccgaattggaacttgaacagtatagccggggtatgggggcgataggccttcccggagatttatcatccgcttcgagattcgtgaaagcagcgtttacaaagatgaactcggtttccggtgattcggaatcggaaagtatcagccagttttttcacattcttggctcggtagagcagcaacgaggctgcgtacatcttggtgaagataaatatgaaataacaatttattcttcctgctgtaacatggacaagggaatttattactatacaacttatgaaaacaaccagatcacagcagttgatatgtataaagaaaatctggacggaaacactataataagctatccgcttatgaaagaacagcagattaactatcggaattactaatacccttaaacataagttttgtaaataatctttgcatagcttttatattattcttttaggtgcgagatagtggtatatctaaaaattttctaatattttgaatttgaacaggctgcctcagaatgaaagtcggggcagcctatttgtgcttgaaataggagcattcctgcgttgcagtaatggcaaataacaagtgtagaataagcaggtactgaattaagagttaaggttatcttatggcacggattatgaagaacagaaaatgtaaaatagccgttgcgatgtttttcgttattctttttttggtgtaccactccttcacgcgggacatgattgcagcggcgaagactgcccgatttatcacttcatacaaatggcactctgacaaattatcggggctttaagtattgctacgacagtggagatattttttgttttctttataaacgaaatgttgaaactatgctttgagcatagagagatttcattcgtagatttgtggttctgccacataatcgacataaagccggtataggcctagccgggacttcgtccctgttttttcataaatcgacgcaatatatcgctgcaaggtgcgacgcgacatataaagggcatccgccatttcctgcacactctcttcggtagaaatcaaccgctcaaatacttcatgctctctctgcgtcagcgaaaaggattctgcaaatttttgagagtattctccccggcttttatttatggatttatccgccaatgtattctttatcacgttattcctgcggtttatatagacaaacattacaacgctgacagccataaacaaaaccagcgcagagataaccgttgcaaagcgatcatctgaaaccagcagcgcaatcgaaaaattagtaatcaaaacagcgctgatattatttgcggtacgccccataccggcccacagtgccggaatcctcatatagcgtgaaagctccatgaaactggcggtaaagaagacgacgaaaaaaccagaagacaagtagaatataaataatccgcccaagaatggaataccgaatttaggaaatacgacacacatgactgacatcaacataatgcagtacattatgataggcatatatttcctgtttttcaaatcaaacagaaaacctgccgtaagcccgcttgctgccagaagaaggcgcggccattgtccgatatccgtacctgccgcgtgttcaagcgtaaccgcgttatcgagcgtactaaaaatacatgccataagcgcaactaacagagcgagaaaaataccggtcgtaagcctgcctgttacccgtgccggagaaacaaccattttttcccgttcattctcttgcgaagcaacaggtttcctgcgcgcccgctctgccctcataaacagcaaggcaagaatactcaaaaaagcagaaagcacaatcgcctctgccaattccgcgttaattagattgttattgataaactgaatcagtattccaagcgcatacgatatccctaccaaacgggcaagacaattgttttcccctattgcacaggcggccatatcgtagaccttgccgctgaatataccaagcagcaagaataaaatgattccgaatatcaacgttgtaaagtaagagacgcactgctggatcacaaagatacatacgactgatgtcattactacgataaacaggaaaatgattcggtatgtttttttgatcaagcggtccagaaatggataaacgaaaaaaccggctgcgcttactcccaatatgtaattttgtgcgttaatagcctctgacggctttgccaaaagagaaaccatatcgacatacaaatattccgcgcccaaaaatacaaaagtaaaaatagccatcatcagtacggcggtcagcgacgctggttttcccagttccgtttttgtttcattccacatacttcgattccttgttaatatcatatctacatcatttattatacattgaaattcagttgattcctaccgtctgaaaacagtttggcgctgttgtgccacatacttttgatagtttggcgctcatgtggcctaccatctcatcatacgaaaagcaataataagggaaagcattttttaagatattcaaacgacaaaaccaatattttaggagggaaaaatgaaacagtgcgataatggtcatttctatgacgaagcaagatttgattcctgtccatattgtcaggaaggcgcaggagtagggaaaacggtagctgcgggcgctatcggaaaaacggtagccgcagtacccggatacggcacaggagaggctgatcgcggaaaaacggtagggatcatccagaaaaaaatcggcatcgatccggcggtcggctttctgatctgcatcaaaggcccgcatcgcggcgaagatttcaagctatgcgctgggagaaactttattggcagggcttctgctatggacgtgtcgctgacagatgacgacacggtttcccgtgaaagccatgcgcttgtgtcatacgacgcgaagcataaccgttttacgctttctcccggtcaggggcgtgggatcacctactgcaacgacgagcaggttgaagtagccaaacctctccatgcatacgatattatagaagttggaaaaagccgcctgctgttcctgccgctatgcggtgaaaatttccgttgggacgacgaagagcagaatgaggattgatcaatggaaaatataattgcatggctgaatacgcctctgacggaggacggctcttttcggctatggatgcttgtcgcggcatgcgctctcgccgttatcatccttttagctttcctaaacaggaagcacagacagaaaaaaacaaaaagaatagaacggaaaacggcttctgttacggagaagccaaaggtaaagcccaatcttgagattgctaatcttcaggggcttggaaaacgggaggagcaacaggacgctttcggtgcaagtctgttgaaaaattatgaggaaaacgggctgcttgcggtgctatgcgatggtatgggggggatggccgaaggcggcatgattgccgggtatgttgtgtctgagctattgcgtatattcccatggcaagattgtagcgaaccggaagagaaaatcaacaggataagttcccatgtttatcgtcagttccgcggacatggaggtacgacgctcgtcgcggcgttggtgaaaggtgggaccttgtctttctggtgcgtgggtgacagcgacctttttttgctgcgcgaagaaaagctctacgcactgaatatgagacaggaatataagaacgatcttttaatccgtgcattagaaggagctttcccgccagaagaggcatttatagatccacaggctgcggcgctttccgaatatattgggaaggaaaccatagtgtgtgaacataaccgcatgccgttgccgcttaggccggaagacacgctgctgctttgttcggacggcgtaagcgatacgctgacattgacgcaggttcgggaagcgatggctctttcgtccgaagaatgttgcaaaaagctggaaagggatattctttcggcagatatcccaaatcaagataattatacggtgattgccatcaggtatcacggaaaaataacggaggagtataaagatggaaacgaaaaagaaaatatcgactaaaaaaattatggcaatcgccttgttctgcgttttcgcgataagctgtgtattgtgcggaatatctgctgccagcagcatgggcatgacacctgctcaagcgcgtaatggcgtggcctatgtgcagcaggtattatatgacgtttccggtagccctgttgcggctggaagcggaacatgctgggctgtcggtaaaccgggagagcccgtggaatattttgtaacaaacgggcatgtggtgcagtatgcgtatatcgttcccaagcaagatccctcgtatagcgggaaggtaacggtagtttattccggtgccgagaatgattttgtagatgcgcaggttttatattattccggcccggaggaaaaggatatcgctattttgaaagttccaagcccgacagacaaacgcatcccgcttcccatcagaccgtctgatactgtgcagccgggagaaacagcttatgctctgggatttccgggtgtatcggatacactggcagagaatatccgctatgatatcgacgacgttaccataacagacggtgttatcagtaagcgcgcgtcggtttcgggaacgggctacgaagcgttccagatgaacgtagacatcaatccgggcaattccggtggacctctggtggacgctgacggcaatgtgattggtgtaaacacgtcggggctcgttactcagatcggtacggaggagcaatcggtaaatatcaacactagcgtaaactacgccagcgttgcggatcagcttatccgtattctggacgaggaacgtatcgaatacacgctgatgggtgaaaaaaactggatgttgtatacctttgtcgtaattggcggtgtaacgcttatcctcggaattgtatttctggttatggacagaaaaaaaacgtcggttctgtcagcggcggataccgagggaagttttgttgccggaaaaccggcaaaaaacagcccgtcaggcaaaaaaccgctgctacgcggcgtcactgggaaatatgccggacagagcttcgatctctccaaaggaaaggttacgcttggacgcgatccaaacctttgtaatattgtgttcgataagagtacgccggggatcagcgggaatcactgccaagtttcctatgatgcagccggggattattttctgttgatggataacggatcttcctacggaacgtttcttggaaatggaaagaaactgcctgcatccgtcgctgaaaagctggcaacgggagatacgttctatctgtgtaacaatgccaataaatttattgttgcaaaggagtagaaaatggcagcatattacttggaaaatgggaaaatacaggaggcatgcgctggatatgcctcccgcgaagacgcggaaatatatcatcttagtgccaacggagaaattacaacaaaagagattgtaccggatctgaaacctggcgaaggtctgctgatgtgtactgaaggtttttatgtggagtcgctggaagtacaagtagacttcctgaaagctgcggatgcggagcattggcttaaatatatggccctgcgccatatcgaacgagcgagatatattgatgacaggctttgggtgttggcagaaatgatggaggaaaaaatatgaatctggaaaaagtatgttacggctgtttccgggaaaaagagccgggagtctgcccatactgcggatatgacgatacagaggaacaaccgtttttagcgctgccgccggggacaattttaaacggacggtatatggcgggaaaggtgctgggagtaggcggtttcgggattacttatcttgggtacgacctgacgctggaaattaaggtagcaatcaaagaatatatgccgtcagggatggcgacgaggtataaagacggatatacggttacactcaccgggcgcgtggaaaaggaatatgcggcaggaatggaacgctttttggatgaagcacgtattctggccaaacttcaaaatttgcctaacatcgtatcggtacaaaattatttcaaggaaaataatactgcgtatttcgtaatggaatatatcgagggaatgagccttaaagaatatgttgcagagcatggaggcagtatttcctatactgaggcgcttgcaatcctgctccctgtcatggaggccctgtgcgaagttcactccctgaatctgatgcaccgggatattagcccggataatatctacattacagcatcaggagaaagcagattgcttgatttcggcgcggcacggttttccatgggtgacaacaaaagcgtatctgttatcttaaaacatggatatgcgccggaggagcaatattccagccacggaaatcaggggccgtggacggatatctacgcgatgggagctacgctttaccgatgtgtaactggaatagtgccgccggactcaatcgagcgcattcacaacgatacgatcaaaaagccctccgagttgggcatcagcttgcctacatcaatggaaaatgcaatattgaaggcactctccgtcaaagcagaggataggtatgctaatatgcgtgcgtttattgatgcgctgtccggtaaaaaagcggtatcggtaacggaacgcgtaacggcaggcgtaacgcaacgtgcctcgacgatagctcctgcggacggtagaagcgacttttgggattttatgaaaacaaatccgttgatacgttggatgattgtaggagctgccgcggctattttagtatttgcggtgattttccctgcggcgggagtatacaatgggaagaacacgcctataacctctaacggtggaacgaatgcccctgccgctacacaggaattggtcgtgccaagcacagagggcagcgccccggtgacaacaccggaaacggacgctccggctatggagatgacaacccatgatattactggttatggcgctgaaatctccgtcccggctgactggacggaagatccggaaagctgggctttcacgagcccggattcgcgtatggctcttggcgttgagctatacttctatcagccctatatggcttgttattccataaacgacgtcgagaacaatgtagaaacgatcgcagcgtgggcatcagaggaaatggggacaacaggatatgaaatcaaaacggcggggccgcaacaaataggagacgtgcctgcctaccagatcgacctccgcgcaacaaagcaaacaggcggagagattgacgtggtactcctttttgctgaaacgcaaaatggttttggattatatgatatcatgggagcttgtacggcagatgatgaagaagcgtacgcagcgcttcgcggatatatggatactttcatgattacaggccctgtcgatacgacgttcaggttgtattcaaaccaagagctgggcttcaaatttctgtatagcgcagattatatacaggaagaaccggctattgttaatgtgaaattcgatgggccggttatatcgtgcgtcgctctatatccttttgcaggagatgaaacacaatttgtagaggtggaaaatgtttctacaattgcggcgactgcacaagaagcgagtaatttcttttataacacccacataaacttgggagcggcgttgaacgagccatatatagatacatggggcggcgttgaatggacaatgtgggaaggaacattacaatcttcttccgttgcattcggtgcagcagagatcaatgggcagatatatgtcgttgccagcagggttcaggaatcaaatgtagagacagcaaatagtgtcagacgtgatgcaatgtcttccctgcgtccggtttgaccggacgcaagggacggaaaggagctatgatatggcagtggtcatcaaacaaacggtaaattgcccgcggcgtatggcggtttatgcgatttttgatacattagataaactaaatggtatttatgatcaggaacttgtgggagatatcaaagcgcaaataaacgtatttgataacttgagtgagtttgcttttgcggtgacggaacagggggatgcctctatcatacatatttccatactctaccctgcgccggaattgacggaggaaggacagcgacgcaccctcgtcttcttgcgggatagtattattcagcatattgagaatgaactcaacaccacagccagagaataaaaatcataaggcagatgtggttgtccgctgtttcggataaacaaacatggaggaaacgaaaatgaacggaaaagataagtgcaggattttaaaggaaatacggcaaaaaatagcagatgagaacgaaatcgaatatgtaacgagggattgtaaatttcagggtgaatgtagaggcacatgtccgcgttgtgaagcggaagtacgatatttggaagaacaacttgaacggcgtaaaaagttgggaaaacgtgtcgccatagccgcactggcgacaggactggtattgggcagtatcggctgtaccgaaagtgactctcggtttcaaagaggctgcgcgcccgtagaaagttatgatgatgaagaatggacgtcgcttgagggagaagttcctgcggacagcttgataaatgaacaaaacgaataaaaaaacggtttttccaacgattgctttggagcgtcatcggatgacaacagatggtattggtgttacgacgcttgttgcggcctacggctgtctactccgatgtcagtattgtctgaatccgcattcgcggcagcaagaaacgaatcaatgcgaatatacgccggaaaaactgttgaatgaggtgaagcaggataatctgtattttttggcaacaggcggcggtattacgtttgggggtggagaaccgttgctttacccggagtttatcaagatgttccgcacactcatgcctgccggatggaaactaaatatagagacttccctttatgccccggcagaagctgttattaccgcggcgcaggtagcggatagtatgattatagacataaaggacatggacaatagaatctacgaacgctataccgggcagaatgccgaagtaatgaagaggaatcttgccatacttcggaatacgatggatatgggaagcgtccgtatccgtgtccccctgattcctaaatataatacggaacacgatcaaaaaaatagcgcatatatacttcaaaagatggggttctccaacttagaatttttccggtatgtcataaagcaaccgggaaggctaaatgcagaataaatgaacataccgaatcaatgcagaatcaaatgattcaaagattactttacgtaggtttccgttttatctgtgatgaagtctgggctttatacagctcggacaacggcaggctgagtattggtattgagccggtggtgttggggaagtatttgctggtgggatacttgcatggcatagagtcgaagcggcgcatcgagcaggagatacaggtaaatatggcctatcgttgacttctggggctggattcagatgagcgggtgccggatcattcaacgatttcataaaacagatgcagcccgcttaatggaacaaatctgttttgtcgcctgcttgagcatatcttgcggcaatgcatagaaaaaggattggttgatggaaagatcactctgccccactccgcccatgcaaaagcggatgcgtcatttaaagtgaatgtcaaagtattggtggaacgtaaaaccatcgattacataaaacggttggatcgatacgaagaagcggagaatatcagaaatgtcggaagttatcaagccccagcgcgtaccgcacgggaaatggaaaccagcattggtgaagaaaacaatcaatacaatatgaccgacccggacgcgggaatgctgagacatccggggaaacctttggggatacattacctgcgtcaccaaagtgtggacgcggcacatgggatcgtggtggacgtggcggtgacggcaggaaatgtaaacgatttggaaccgtaccttgagcgtgtggaatatatgtgtaaccacatcggattaaatattcaggacacgggagctgacacgagttatggaactagcctgatctatcacgaaatgaagcgcatgggaatcaggctgcacacacccaaatcaacagacggtgagacatacaaggcagagctaaaacgtgaacattttcgatacgatgatgaagagaatgattactttgtttgcccataagccggatattgaaattgaagagtctggagcgagagtgctacaatatctgcaaggtctatcgggcgatccgcaaagattgttgagattgcgtgatactggagcaatgcgtaagcagtagccaacatagccggagcatccgggtaaatatctttgaagaagcggtgagtcagtaacggaagacagacgacacgcctgcccataaacgagttttagatttaagacagatatggtgcgagggaaattttgtggtgcaggagttgatacacaggttaaagtgcctgacaaggcgaggtaaggaagcggcagaagaccattgcctcctatccgctactgccttgaacatcaaacgcatgataaggtatctgggataactcctatgtcattattttttgctttgattttctaagtatcatcacttcgcctgagaaaaaagaaatagagaaaaaggccaatggctatgataaagatgtgggatttctacctgcggtacacagaagaatgtgtggaagtaccggacaggatcgtggaggcgatgtatgggcacaagcgtcaggaggaatccggcgaacggtatatctgccgttatcgcgcaaaaaagtgtatcaagctttaggggaggtctaaaagaatgtatttgcgctaatgatgtttgataagtatacaaattcaagctgtgaatcgggagaaagctaaagcatggtcaaaagaaaaaatacaaaaaagagaaaaaatcagcctcattaaggggtaaatagtccagaaaacaacaagacgtttaccaatattacaaatagaagattgaacgcaagtaaaatatttgtcatattgaagctcttgaaatttacgtttagacattgtattataaagaagtaaaaattgtttttagaagcggattatattccgcttttttgtaaaaagaattctgtgcggcaagaggcatcagcaggtaccattataattcattcacgctacaaaatttgctgtaaaaatagataacaagtatagattattagaaaaaagcattgcaaagctaagtggtttctttcatatgggggctaattagttggtaaaaaagaaaccaactttgcacaggtagacggttttttatttgtgaggtaaaggaggcctcatgggaaagaaaattcggcgctttttgtggagtagcttggtaggcatattaatcttatgtattgctatatttacttggataactttctatatggtcaatgaaaatgacaggacgattagtgatatagggaaaatttatatgtccgaaatgggcaagcaaatctcgttgcatttttccactgtaattgatctgtaccagtccaagctgaaaggcgttgtatggagcaatatgcgcaatgatataggcgcggcgaaactgcgagagaatttggctgccagtgcccaaagccttggattcagttatcttggcctttatgctgatgacggcagctacgaagctatttatgggaacccatacagatagacgaagaaagtgcctttatggatgcgctaagcgcagacgtgcaaaaggtaacagaaggcacgaaccagtcaggcgatgatcttcttttactgggaacggctgtttcttttcccatggaaaatggtgaaaatagcactgcgcttgttgcaggtatacccatacagactcttatagatatactctcgctcgatatcggtgaaacccgtgtttattctcatatcatccgcaatgacgggacatatgtaataaaaaatgccgatgcaacagaagaagattccgattcgtatttcgcccgtgtgctcaattacggtcatttcggcaatggtacccctgaggaagaagtacagaagatctcagaagcgatagaagcgggggagaaatattccatggttgcagagatcaaagatgaaatccgcaatacacattttaccccgctgggatattccgactggtatttggtatcagttcttccttatgaattgctgcatgaacctatatctcatctgcttgatcaacgtatttttactgccatcagtggctgtgtcatcattttatcggtaatgcttttaatatattataagtttttccgaatgtcccagcgacagataaagcttttacaagagacacggcaggaagcggagcgtgcaaatagagccaaaagtacatttttttcaagtatgagccatgatatgcgaacgccgatgaacgccattatgggaatgacagctatagcttcgacacatcttgacaaccggatacaattgcaggactgcttgaaaaaattgccctgtctagcaagcatctgctggggttgatcaatgatgtgttagatatgtcgaagattgagagcggcaagatgatgctgaatgtggatatactttctctgcgtgaagtgatggataatattacaaatatagtgcagtcccagatcaaggcaaagaaacagatattcgatatttttattaaaaacatacagactgaacatgtatactgcgacggcacgcgactgacccaggtgcttttaaacatactttccaatgcaatcaagtttacgccggagagaggaactattcatgtagcattgacacaagaggattcgccgttaggagaattctatgtgcgcaaccattttggggtgagagacactggaatcggtatgtcgcctgaatttcaaaagaatattttcgattctttcgcgcgtgaggataccaagcgcgtacagaaagtcgaaggaacgggtcttggcatggcgattaccaagcatattgtcgacgcgatgaaaggagcgatagaattaaaaagcgaagtaaataaaggcacggaattccacattattttggatatggaaaggaatacggaaaaagaagaagatatgcttctctcggcttggaatgtgctggtggtagacgatgacgagcagctttgccacagtgccgcttcttcgttaaaaaagattggcgtgaaggtggattatgccttagacggcgagactgccgttgaaatagcggcgacgcaccataaagagaaacagggttgccagattgtattactggactggaaaatgccgggaatggatgggattgtgactgcttacgattggaatgaaatcgaggaagaagcgcgcaaggctggagtcaacggtttcctttccaagccgctatttaaatctacacttttctacgggctcaggccttttataaagactaccctgctggaatcaaagccgctggagaaaaggattaatttcatcggtatgctcggcgacacaacggccacgggctttgtgcttgcggttatgatgtccaactcaggcagcgcgtgggataacgcgaagaaatatatcgagggcggtgcgcacggcggaaaaggcagtgaagcgcataaggccgccgttgtcggcgatacggtaggcgatcctttcaaggatacgcccggctcgtcccttaatattctgattaagctgatgtccatggtggcggtcgtttttacggcggttatcgtaaattacggtatactacaataaattcgaccttcagagtattaaatatattataaacaatatattcttaaaaaaatacatataaaatatagcacttttgcaatcttaaaccgcaaaggtgctttttgtatgtccttttatagggcacgagcacctccatttctccattttgatcgggcgttttcaaacaagcgacaaaatggaggttactatgaaaaaagtgaatctaagggagttttatccatctctctatacgaaagatacttatgtctttctaccggatgatgtagttactgctttatgggaagaaacaaaaagagaaagggcgggatatgcaaaaatacggtatcaccgcgcatactattctcttgaccgggaggatgggattgagcgatttgccctgcactcccctatgctgccggaagagatcatgcagcaaaaggaactgcgcgaatttctttattctatcattatggaattaccggaaaagcagctgaaaaggttctacgcctactatgtactgagaatgaaactgaaagatattgcgcggattgaaggaaccgatgccgctgctgtattacggtctgttcaaaagtcagaaaaaacgatccgcaaaaaaataaaaaattttttagaagattgagacaaaaaacggtgtgtttttctactgaataataagggagtatttttcttccgataaaaacgaaatatccaatcgtctgaatacgttagctgcatagcgagcaaaagaacgcacacgcgaggactgcctgcgggaatattcccgtcaataagataacatcaaaggcacgagcgggaatgtcaacgtccggaaacttccgtggctgggagtatgccatgacctatacagcctatcatgatacttgcgcgtagtcgagatcaagttccagagcgtgcccctgccgttggcagggtggtgagattccaatgtggcgctgcccgcgccggttcagacggttgcccgtccgttatgggaagtagtgtcgaatgataacggaatagaatatggaaagtgggggcagattttctgatctgaatatcagcacgaatagtctgccccaacttctgaatttatcaaacctctttgttgtaatttggaaatatgagttttttttgcatggagattgtttcggactataataagagcatccgccaaatctttgaaaggaggtgcctatttgtacgaaacaaagttgccatgtgagacctgcaaagaaaagtcaagccggaaatgaataaaaaatgaaaggtaaaaaatggaacatcaaataaaaccaccctgaaataagctggtaagccaagcaaaaagaatatgtgggctactgaaccgccaaactgcctagatgctccttaacccgtgcgtagtagatacgcaggaacttgttggctcctgccatcatgtagacgtaataatgcttaccctcgccgcgttttttgtcaagatattggaatacagcatcatgagagggcgcgtgtttgagcaggcagtccatgacctgaaaaagcgtctttctgagtgaggaagaacctcgtttggaaatgccggtactctgtgcttcaaaggcgccggactggaaaggcggtgcgtcgataccggcaaaagcaaccagcgactgcttacggttgaaacgtcgcacatcgccaatttcagccatgagttgaggtcctaacacattgccgaccccttgcatagccatgacaacaggaaattccggcaagccggaggcaagggaaagcatctgctgccgaatcagagccagcgtttccgatagggagttaagctgcgacacagcttgagtaataagctgtttggcaaaggtgcttcgaggcagtgtactgacttgtatgcaggcgaaatcgtagatggcagcggccttagtggaaagaaagttgtaacctgttcttttgcaccatttttgatagcgctgggtaaaaacgtttttggaaaggctgttgacgcattcaaaatgccaaaatttacccacgaagtcaatccatttttcgtgaccgtcccccttgcggggcgggctggagaacagcatgttgacactcggaaaggtttggtcaagcaaagcgatgaggctgtttttaagcataacactctgcttaacgtactggttgcactggcgggcgcaggttttaagcatgtggcgaacgtcttcttccggcgcatattgcggaagatcaagccaatggcttagagcatagctggcgattttaacagcatctatcttgtccgttttgacccgtctaagggaattttgaccgtagtcatgtacaagaatagcattgacaacggacacatagaggccggcttcgtgcagatgcctggcgacaggcagatagtaattgccagtatgctccataacaatgcgggtttcaccgtccaaacttttcagacgttcaaccaaacgtctgagttcatgagcggtgtgctgaatttcaaaaggtgaagcaaccacctcaccgaagggacgcaggatagcaaccatgcacttgcccttggaaatgtcgataccgacagcgttcataagtattcctcctgattgatgtattgtaatcggtgcccatctcttacccattgcctattcaatctactgagtgacacgaacgcacgagtggtggctctacctgcaaaacgaacgctgcaacaagaggatggctgactgtctcacgtacgggcgtgtagacccaagaaaggagtcgtcagaccaattactccccttattgtagcttagactatgggatggaaaggcgtatggctggctgccatatcctatccataaattattgtagtagagaaaggagagtgcatttatggaaaatctttcctctcgcgtctatacggtcaaagaagtagcaaaaatgttaaatatgaagccgaggaccgcttatgccttttgtgaatccacagataaatttcaagtagaacgtgtagggcaaagagggcttcgtatcaacaaagcatcatttgacgcatggatgcccagcggaacccaatcggctaatacgaaagaggtatagaaaatggcaatttcaataaaacaaagaggtaaaaaatatacgttcacattctattataaagacgaaaatggaataaacaggcaacaatatgaagcatatagcaacctggataaagccgatgaacgagcaaagatgattgaatttctgaaatggaagaatgatgataaaacgctgctacagttagtaaatgagtataatgcgaataaagcaggaacaaaaacaaagcattattctaaaaatgataatatgtgcaaaacattctcagagtttatttgggcatgggcacagcggcatgcgcataaagtacgaatgaagccaagttcgtacgcaggtttgaagcggaatattaggaatcacatcgagccatatttcggaaaaatgattgtgagcaagattacaccggaaacaatagaagattttttctttcaattaagccaaaagaaatgcaggggcgcaaagagttataaaaagagtgaagatcaaatacctactcttaaaggaagttcgctaatgaaagtatttaatattatccggcctgcttttcgggatgcggaaacatggggatatacgaagcgaaatccaataagcagtgatcaagccccggcatataagtatgcaaaacgaaaattctggaaaaaagaacaggtgcagcatgctttggaaaaaattaatgatcccgttttgcatttagctgtacatcttgcatttatgtgcaatatgcgaccgggcgaagttgtgggaacggatgctgaaggtattgatttggcaaataaatacttccaaatcagtcaagcgcttcagcgtgttgaaacagaagcccttgaagagcttgctcaagatgatattatccgtattttcccgaaaaagaaaccggacgcaaagactgtgatgatcctaaaaaagccaaagacagaaaaaagtgagcgcaaaggattcctaaacatgaaactgtgtgaggaaatcgagaaacgtttgcaacagatcgagcaagataagcgatattacggcaaggattatcatgactatggcttgctcctttgtttcccaaacggtgatccgattgaaccccggacaatggataagcgatttaagaagtggcaaaaaagtgatggggtgagtgcggaagatgtaattgatatgcaaggattacgaaaatctgcaacaatgtataaattgaggttaacaaactatgatgtgcaggttgtcggaggcgaaggcggacagacatcggctacaacgattcttgaccactatgacgaagttctcgaagaagatagaagaaagttatctatacaagtggaaaatgatttttatgggcggctgaaacaaaaacgggtaacagaggactttgacttgcgaaggttatttaaggagttagtcgaacagccggatttagcaaaaaaactttacgcttgtttacccgtgttgcagaaagcgacctgaattgcactcaaaatttatgagcaaaatttatgagcaaatgatttttgctcataaataatagatattggcagaacaaaaatagtagaaaaactgaagacctcgataaagatggtaatggaaaagcggtttcctacaaactcgtaaaaaaccgctctaccaaaggatttctttggagctgatggcgggactcgaacccgcgacctataccttaccaaggtattgcgctaccgcctgtgccacatcagcattatgaaatttttaggtgaaacgcaaatccttaccaaggatgtgctctacccctgagccacatcagcataataaaactgtgacttaaatattatatgatactccggcagatttttcaatccctttcaacggaagaaattttcgcttgcccctgggggtaaaaacatgtaccaaactattaaaaaagcataaatactttcacattttgtttttcttacgctataatgaaattattctgaattaattttatgaaaccctcccgtttcataagaaataaagggaaagaaggattttacaatgatttgtaacaactgtaagcaaagctacgacgacaatttgaacgcatgtccttattgcggcgcagccaaggcggttactgataacataccgccgcagcaatacggacaggtcccgccgcaggcataccaggctaattctccggcaaagggtacggcgacgggcagcctggtctgcggtatcatcgccatcgcgataggcgttttcgtcccgctcgcgggcctcattctcggcattattgcgcttaccctggggaataaggccaagtgcatgctcccggcggaccaaagaggtatggcaacggcgggctttatctgcggtatcatcggcatcgtaatcgcggtcatcttttggatcataaatgcagttgtggcggcttccatgttgtcttacttatactattgatcggagtgtaaaaatatcatgagaaataaacgcggaatttttgctttactcttactgttcatgatattgcttgcagccggatgcgcgtccagcaccgctccgacggttgaaataggcggcgaggtcctcccctccttatattccgtggtgggagaaaagaaaattaccggcacatcgaaaagtatcagcacgggacttgcggaggtagagcttacctatcagggcgtatcaatcgacgagctcaattcctatatcgagtctctgatttcggacggttacctggttacgcaggaagcgtcggcaacaggaagcggccagggctaccagattggaaaggaagcatccacagagggaaacatcattcttatcagcttttactttgaagacggcgggtctacggtcatcaattatgctgtaggcgcgggcaccattacgccggaaaccagaggctaacatttcccgctgaaaacagtctcccaaaaccgcaccgaggcgctgccgcggtgcggtttctatttcggcactgcgattatttttgtttggtccggcccttttttccgtataataaaagaaacaaatcctttgggaaggagcctccatgattttacattacgagaaagaacgcgtttatgcgaaagatgaaaacggaatgctgctggccgagatcacattcccggagacagcggacggatgggcggatatagaccatacctttgtcgatccttccctgcgtgggcagggcttagccgataaactcgtacgcgccgccctccagcagatcgcagaggccggaaaacggcccgtcgccacatgcccttatgcggtcaattggtttgaacagcatccggaggaattaaaagatttgttcggcccataaaaaggcttttaaatgggtgcttttgaacagcgcggccataaccaaacattttttttgaaaattttaaaaaatacttgcaggattttgaattcgggtgtagaatacatataataagaagatcatacaaggaatcagtaaaaaatggtttgaggaactgatttcagggtatgataatagtaaagacagatggacttcggcggatggtggtccggaagaacgtctgaatcgaaaaagaaagggcgtgataccaatgtacgattccgaatgcgtgacgttgggcacgcacgcacagtcccagaagtaggctttaaggtcgcacgaaagggatcacggaacaggatttttgaattggcagttgacggtttgaaaatatccgtgacagtggagcaaggttcccggagatgttgtgaagaaagcggagggaaaaaccggattccacggcagagttgtaaagaaagctcagtggaggcgccgaaaaagcaaacgtatcgcgggcgttgtagccgcaagggagtctggaacaattccagaccaaatgtgtgtggtgccgaaggtgtagcttgttcgcaagaaatgcggtaccggagcaaccggttggtacgtaaatacaaatactgcgtatcaatgtggcggtcggcagaaaccgatgggggatgcatgacccgaaggtataggcagagcgcaggcgcacaaaagtaaaaataaatgtaaatatggcaatggggtttccgaatggaaaccccattttttcgttttacgataccgcttcccgattcgcctcttcctcgttcttttcgcgcgaaatcctgatgtggagtttgcgctccagatcttttattttcccgtgctcacgcggcgtaatcagcgtatatgcttttccatccttgcccgcgcgtccggttcgcccgatacggtgtacgtaataatccggtttgtccggcacgtcataattgatgaccagatcgatcttatcgatatcgattccccgcgccgcgacatccgttgcgatcagcacccttgtttttcctttgcggaaggtattcatgatcgtatcccgttccttttgccgcatatcgccgtgcaggcaggccgacgcgagtcccctatggaaaagctctttttgcagctctttcaccttacgcttggtgttacaaaagacaagcgtaagccggggagcgcaccggccgatcagcccgcataccgcatttgatttccgtttggtctgcatataggcctgcgagaccgtttcgaccgggcggttctgtcccccgatcttaacacggaccgggtctttctggaactgccccgcaatcgccgtgatttccttgttcatcgttgccgagaacagcatggtctggcgctgctccggcacagcggacaaaatttctttcatatctccgagaaatccgtaatccagcatttcgtcggcttcgtccatcacgaccgtgcgcacatttttcagcttcagcaccttgcggttgataaggtctttcagacgcccgggcgtaccgacaacgatctgcgcgcctgtccgcagggctttgatctggattcccgcaggctgtccgccgtaaacgctcacaatccttacccctttcaggtgggccgaaagttcagccgcaaccttagcggtctgcatcgcaagctcgcgtgtcgggcacaggatcaatgcctgcaccttcctgttgggcggaaaaatattctccaggatcgggatcataaacgccgccgtcttccctgttcccgtttgcgcaaggccgatcagatcctttttttccaatagcgccggaagcgtctgctcctgaatcggcgtcggttttgtgtaccctatccggtaaagcgttttgttcatttcttttgataactgcatcgaaaaactcaaataactacctctttcccactccggcaaaaaataagactttgcctttcaaagcaaagtcttttcatcaatctactttgtgtacgtctgaagtatagcacactttcttccatttgtaaaatggaagaaagcttcagctgtccctccttaccatattctgcaaaacaggactgcttataacttattttgcatattggctcagatatcaaggaccactttctgaacccgatcttgattttgctcgatataccggaacgcatccgccatcctgtccaggccaaacttttgtgtaatcagtcttttcaggcatattttcccctgctttaccaggtcaatcgattccatataatcttcctctacatacatcaaagtccccaaaagcgtatattcacggtcctgcacatttgccagattgatatgcggcatcccagcaaaaacacctgcgaccacaatcgggatccctttcggcgagaggaaaagcgcttgattaagtgcggcttcatttgccgtgcattcatagaccgcgtctatcccatcttttccaaacactttttctatttcttcggcaagctcctgccgggatatattcacagtatgcccgatcccgcattgttttgcaagatcaagccggaaatccgagatgtcggtaatcattaccttcctggctcccattccttttacgctttgcgcaaccagattgccaatcgttcccgcgccgaggacaaggatattttttccttccacttcccgaaacgccctttttacagcatgtaccgcaaccgcaagcggttctatcaacgttccttcgtcaaaaccgacgttatccggcaggcggtaaaataaatcctcatccgcgacataatagtccgaagctccccccgggcattggcagccaagcacttccagcttttcacaaatattgtatctgcctgcccggcagggtttgcaggctccgcataccctctgcggccgtaaaacaacccggtcgccttttttcagctttgtattttcgcctgaaatttcttctataattccggtacactcatgtcctaacacaatcgggaagtccataaacggatgctctccgtaataggcatggatatcagagccgcatataccgatggctttgaccgcgattttagcctgtcccggacctatcgcaggctgttcgacttcgcggcattcaaaatttttaatcccggttaattctacctgtcgcatctttcgcctcccttaaaaatcaaccactactttaatagaatcctttttgccggcaaccgctttgtcgaaagcttcttgtgccttttcaaacggaaactgatgtgtcatgaccggaaggatatttgcccgcccataatggatcagtgtcatcgctttctcaaactggttcacataacgaatcactgttttataagtaaggccgcgcataaccgcactcgacatcggcataggggtcccgtcgtcgatcaaaaggcccagtaatgttatcgtcgccccgtcccgcacatatcctgatatatcatagaatgtcttctcggttccaaccgactcaaacgcatatcgcgccccataaccatccgttaactcccggattttttccgcaacatccccatccctgggattgatggtatctgtcgctcccattttcttggccatttcaagcttaaacggctccatgtcgactgcaataatggttccggcgccaattgttttaagcgcatgtatggtcatcattccaatggttccgcagccgacaacgaccgcgctgtccgcgtatccgatcccgctgttggtcacgccctgcagcccgacggtgaacggttcgattaacgcaccttcctgataggacatatcctccggcatttcatataccaaaaattcgggccacgctacatactcgcacatgcatccatcccaaggcggcgtacccataaaatacatattatggcaaaaattataccggcccgcgcggcattcgtcgcagcgcatacacggttttcccggttccacaatcacccggtcgcccacggacaggcggctgcatccttccccggccttgacaacctcacctgcgcattcatgcccgagaatcaacggttttccgttcatggcataatcgccgatttttccatccgtataaaaatgcaaatccgagccgcatatccccatggatttaatgcgcaccaatacttctccctcttttacgtcgggcatcggcaaagcccgtatttccatcttccgcggctctgtcagcacccatgctctcattgtttccattgatatccacctcattatttaaagtttgtttttccgcgcttccttatcactcctgtcaggccgccatttcatttttggccaatttccctctccgcacattatccagcaggagtgccagaagaataatcgctccctttgcaacatcgcgcaggaatggatttacgttctggagagtcatccagtttgagatgacctcgataatcaatgcccctgcaaccgcgccaataatatagccgttgccccccgaaaggctcgtcccgccgataacaaccgccgcgattgcgtccagctcaaggccttctccgagggttgcggacgcagacataagtttggagctcaaaataacagaactgattgccgaagtaatccccgcaatcacgtaggcaacaattgtagtctttgcaatactgattccggagtaccacgcccccttcgcattgctgccaacagccagaatgcgcctgccaatcgtaaaccgcttcatcaggaggattgctactacggttaccgccgcaaatatccagatcggcgtcggaacctcccccagcttgcccaagccgaagaaatcaaagccttccgccacaattgggaattggcgtcccgatgtgatgtagagtgcaattccatacgtaaccagctgcatcgccagcgtgaccataaagggcggaaaattcaactttgaaatcatcaggccgttgcaggctccaatcgcaatccccgttgctacggcaatcaatatgacgacaggaatagataggcccgcggaaaagcataacgcgctgataattcctataatcgtaacttgtccgccgacagaaaggtcaatacctcccgtagcgataacaaaggtcattccaatcgccagaattccggtagtagctgtccgctgcaaaacgctgaccatattcgttggagaggcgaaaagcggcgttactatcgtgactacgaggcacagtacaaccaatatcaaaataattgaaaactcggatgccaatatcttttttactttttctctcgtcatttgtttgtcctaccttacatatagatttgcatttttgctttgtctatttctcttgccatgaacgtccccgcaatctttccctctttcagtatgattattttatccgacatgttgagaacctcttctatttctgacgacataacgacgactcccactccttgttccacgagggcattcatcagttcgtataccgcaactttggcgcctacatctatgcctcgcgtaggctcgtcgaatacgacgatctttgattgggccgctatccactttgcgagtacaaccttttgctggttcccccccgaaaggctactgaccggatattcgaccccggcgcacttaatttgcagggctgcaaccatttcatccataatgccttttacttttttccggttaatccatcccagtctggaaacatcctttagggacgcgaggattatattatctaaaatgctcatttccgtaacgagcccctcttccgcccggttttccggaaccagccctatttttctggcaattgcctccgacggctgccgggcggtaatttttttgctatcaaggtaaatatctcctgccacgcggggatgccccttgccaaaaacgccgaacaacgccttggctgtttcggtctttccggcccccaaaagtcctgcgatgcccaggatttctccccggtgtaccgaaaatgaaatgtcctccagcagcccttctacggagatatgctcaaaacgcagtaattcttcccccctggggaatacctttttcggatatttattcttcagcttccgccctgcgataagttctgccagtttttcttcgtccagttccgccgacccgtattgcccgattttctgcccattccgaaatacgatgatctcatccgtcacttcgaggacttcttggagcttatgggaaatatatataacgatatatccgaggtcttttagccgcttgacagcacgaataacggcttttccgtctttatccgtaagcgcagacgtcggttcatccaaaataaccaccttgcagttctgtgagagcgcacgcacgatcgcgaccagctgctgatgggcaaccttcaggtctcccaggcgcgtatgtgtgtcgatatcgatttccatcatgtgcagtaattctttcgcttgtttatgaatttttttccaattgatatagcctgctttgctgcccgttaaatgagagataaagatattttccgcgacgcttaaatcggggagcatattgagctcctgatatacaatccctacccccaaatgctgtgcgtccagcggggatgaaatttttaccggtgttccatctgcaagaatctttcctttatcggcaatgtaggctccgttgataatcttcattgtggtagactttcccgcaccgttttcgccggctaagccataaaccattccgctgtgcagccggaaactgatatcatccagagctttaatacccccaaagcttttacagatcccttttagctccaggctttcctgtatcatctatattcaccactttctcccgatatccgtcttgtttccctgttcccatcagggcggcgatatacgcaccgccgccccgcttgcaaaattgcttcaattcataaagctattccagatatcttccgtaccagaccacagctctgtctccgtttttacatcatacagcgcccacagggtcgggtcgcttgtatccgcgcctgtaatgacccgggtttccatagggatcacttcgccgtccagtgtctctcctttagaaacgatctcaagacatcctttcgccgctactgtcgccagcgcaagcggactctgttccacgcacacatccatcaaaccggaattcagggcgtctacgccgctgggatcacaatcgattccagaccagtagatatgattctcctcgccgcctttcccggtttttccgttttggcgcagggcggaaatcacgccggggtccatagagtcacagtgcgtaataacaccgaacagatccggattcgccgttaaaacgtcatttaacgacttttccgccgtatcgcttgcccagtcgcatggactttcaatcagctgcattcccgtttcggccgctacctcgtcgattccatcctgccggagataggcgtcactctgcgtcatcgttccctgcatgacggctaccttcggattttcaacgcccaattcttttgctttatcagccagccactgcgccgcactcgcacctgtggccacattatcgctcgatacgaccgcaaaattgaggttgtcgcctgaaatttctttattaaaaccgatgcactgtattcccgcatttaccgctttttgtactgccggagccagcgcttcgctgtctgctgcatggatcgcgataatgtcacagcccatattgataaaatcatcgatttggctggattgttcgtttgcatttccatcgcagttgactaacgtaacttccgcacctgcggtctccattaatttcacaaaatactcggctgcgcttacaatataggtggacgataggtcgctgaccgttaatccgacttttagtccatcgcctttgcccgccagctctgcggcaaaaccattgcctgccgccgcgccgtccccttcggaaacctgggcagattcttctgccggggctgccgcgctttgttcagcggtttcgaccggctccgcagatgtctgcgctgccggagctgcacacgcggcaagcattgctgccgccagtaaaagtgccaccacaaccattgataattttttcatgttctcctcctgatatattggattattttcttttgccccaatgaggcaaacttaatttttttcgatagattccggacattcctgctttttcaaagcccgccgtaccgagatacgccggtctccgatcaactatgcggtcacataggtgtgaaattctagccattagaaaaaggcacaaaaaaaggcgccttttccagcgccgataaataaaaatacaaaaaaaacgattggttaaaaaactacacttttttcaccaacgttccgtatattctgatttcataaagctttgatacagggtagtataaatagccgatatcctaaacaggatcgttttaaagttaatcccctttgcttgtactttaacacagtccaatatacatgtcaatattcttttagcgcgtccattctcctcctgcgctggcctgctgcccacaaaattgcataatctgcgcctttttgccattttttccgctttttggatcttccggtaaaattttatgccctgaggttttgtcttcaaaggtatcttctgtgggcaagggtttgacaaagatgattatctataatataatcctgtgtgtagtagaagccaatcgaaaacattcctgtatctttgcatgtaaaggataaatttttatcctgcagcaaatctttatttgtttttcatcttttttcgatagagaccggattttgccgggcttttgttttatcatgctttcttttgaggattcatgccgggggcgcggtaatacgcatgcattttatacagaacatacatattacaaaaggagattctaatttattatgattagcaaaccatattcaacagatttcaggctcgacggaaaaattgcactgatcacaggtgccgcttcgggcatcggtctcgctatcgcccagctcttttcggaacagggcgctactgtatgtcttctcgatcggaatataaaaagcgcgcttggagcttcggccaacatccccggttcttatgcttacggtgtggatattgccgattcggcatccatcgaagaggcgatcagccaaattctaaaagcccacgggagaatcgacattctcgtaaacagcgcggggatcggtacggtcgaatgggcgaaggatttcccggaagaagattggcgcagcgtaatcgatataaacctgaccgggaccttcctgatatctcagcgggtaggccgtgaaatgattgccatgcaggagggcggaaaaattatttgcctcgcttctcaggcaggaattgttgccatcgacaagcatgtcgcttacagtgccagcaaggcaggcatcatctctatggtgaaatcccttggctatgaatggggaaaatatggcattcaggtgaatgcgatctctcccactgctacggaaacgccaatcatcgtcggatattgggatgtcggcaaggttcatgaagatgcgatcgccaatactcccgccgggcgcttttgcaagcccatggaagttgcgctggcagccctgttcctggcctgcggcgcttccaatatgattacgggctccaatctcgtaatcgacggcggttataccattcattaacggtaccgttcctcctcccaggcgtgttctgtgccgtctgtccggaatacgcctgggatagaatatccaaacctattgcatttatcttcttacaatccgctgtccaatagtatgcgcagtgtcaggaggtctctatgacaaaaatacccgaataccggaaaatatattccgaactgaaaagggaaatcaagcgtggcacctatagcccagggatgtttctgcccaccgaaacggagttggaaaacatgttcggcgtaagcagaactaccatacgaaaagcaatcagccttttggtactcgaaggattcatttccgtacagcaggggcgcggaacggaagttcaggaagtctccacatcgcaaaggctgaaccagatcacttccttttcggaaaccttgtctcaaaagggctatatcgtaactacgcgcggattttacctcgaacatattcccgcgcctgcgtttgtccgcgaagcgcttggattggagaaagattgcacggttcatcatttgcaaagagtgcaatgtgccgatgggcgcccgatttgtattatggagaattatatcagtgcccatataatccccgatttcgagattcgcaacagcaattttgtcagcctgtacgagcttttggaacatgaatataatctcgttctgcgtgaggctgttgaacgtattaccgccataggcgcctccttcacagaatctcaaatacttcttgttccttcaggcaccccactcttattcagcaagcgtatcacctactgtgataaaggaccttttgaatatgtaattaacaaagtgcttgcagagcagtatgaatatgcaattcatctttcgggaagggcttaggacctaattttccagcttggtcccagaaaaatatgattcgataaaaaaaccgccccagccaaatggctggggcggtttttacctataaatgattgatttattttccttccgcgaggttcttgtagtattcgtaccgttccttcgcttcctcttcggattttgcaaacagcttgtccgccatttccgggaagagtttcttcaaggatttatagcgggtctcgctcatgatgaaatcctggaagcttgccgttgccggtttggaatcgagcgtgaacggattcttaccctcttccttgagcgccgggttgtaacggtacagctgccagtagcctgcttccaccgcgcgcttttcttcgtgttgtgatttgcccatgttgataccgtggttaatgcacggagcatacgcgatgatgagggacggacccggataggcctccgcttcctgcatcgccttcaaaagctggccctggttcgcgcccattgcaacgcttgcaacatagacatagccataggtcatagcgatcgcgccaaggtctttcttcttgaccttctttccggatgctgcaaacttcgcgatggaaccggtaggcgtcgccttacttgcctggccgcccgtgttggaataaacttccgtatcgagcacgagcacgttcacgtcttcgccggaagcgagcacgtggtcgaggccgccgtagccgatatcatatgcccagccgtcgccgccgaagatccagacggacttcttaatgaacaggtctttctggtccaatatcttcttgcaggtatcgcactccgccgcaaccttcccaagggccgcctcgaggttcgcagaagcggcctttgactcgtcgcccttgtctgctgcttcaagccatgctttcattgcttccacaacttccgcgtccttctcaccgggcgcgtcgatcgcagcctgcacctgctctttgagggcgttcctgcgggccgtgaaggcaaggttcataccatagccgaactccgcgttgtcttcaaacagcgagtttgcccaagccgggcctttgcccgcgtcgtttaccgtataggggcacgtcggagcgctgccgccgtagatggacgagcagcctgtcgcgttcgcaatcgtcatcctgtcgccaaagagctgggtaacgagttttacatacggcgtttcgccgcagcctgcacatgcgcccgagaactcgaacagcggctttaggaactggctttccggaaccgtattctttttgatctcgatcttcggttcgggcagcgccgctgcaaagtcccagttctctttctggagtttttcctgctctgcaaggggcttcataataagggccttttcctttgcggggcaaatatccgcgcagttgccgcagcccgtacagtcgagcgtagaaacctgcatacgatagttgtagccggccttgccgcgtgcgggcttcgtcgcatagttcgccggcgcgttcttcatgtcttcgtctgtcgcgaggaacgggcggattgccgcgtgcgggcatacatacgagcactggttacacataatgcagttatcaatctgccactccggaaccttgaccgcaatgccgcgcttctcgaatttcgtcgttcccgtcggaacgaagccgcgcggatcgaaggcggaaaccggaagctcgtcgcccttctgcacaaggatcggatggatcacgtcgcagtaatatttgtcgtcgggaagcgtcaggcagggagcgccctgcgtcgtggccgcccagctttccggatagtttacttcctcaacagccatgcctgcgtcgaccgctttgtagttcatgtttacaatctcgtcgcccttcttgccgtacgttttcttgattgcctgcttcatgtactcgatggcgtcagcctccgggatgatgcccgcgatcttgaagaatgcagactgcataaccatgttggtcctgccgccgaggccgatctcgcccgcgatcttgattgcgtcgatattataaaatttcagtttcttcttggcgattacgttcttcatgaccgccggaagctccgtttccatctcttcgagggaccagggggagttgaggaggaacgtgccgccttccttgattccttcgagcacgtcataacggatgacgtaggacggattgtggcatgcaaccatatcgggatgcgtaatgaggtatgccgactggatcggggatttaccgaaacgcaggtgcgaaatcgtgatgccgcccgacttcttggagtcgtaggagaagtaaccctgcgcatacatatccgtatggtcgccgatgatcttgatagagttcttgtttgcgcctaccgtaccgtcggaaccgaggccgtagaatttcgcgctaaagaggccgtgcggcgaagaatcgatcatgtctttcacttcgagggacgttcccgtcacatcgtctgtgataccgaccgtgaagtggttcttcggcgtatcctgcttcatattgtcataaaccgctttcaccatggaaggcgtatattctttggagccaaggccatagcggcccgcataaaccttgatacccgcccggcctgcttcaaagagcgccgttacgacgtcttcatacagcggttcgccaagagaaccgggttctttggtcctgtcgaggacggtcactttttcagccgaagccggaatcgcatccacaaacgcgcttgcatcccacggcctatacaggcggactttaatgacaccgagtttttcgccctgcgcgttgaggtagttgacggcttcttccgttacgtcgcagccgctgcccatgataacgacaacgtgcttcgcatccggtgcgccgacataatcaaacaggtgatactggcggccgaacactttcgcaaattcgttcatcgtttcctgaacgattgccggcgtcgccacgtaatacttgtttgcagcttccctgttctggaagtagatatccgggttctgcgccgtaccctgctggtgcggatgatccgggttcatgccacgtgcgcggaactcctcgatcgctttccagtcaaccatctcggccagtttgtcatagtcgaggagctcgattttggaaacttcgtgggacgtcctgaagccatcgaagaagtgcacgaacggaaccttcgctttcagcgtggaaagatgggccaccgcagccatatccatcgtttcctgtacggaagcggcagccagcatggcaaaaccggtctggcgcgcagccattacgtcggcatggtcgccgaagatggaaagcgcgtgtgccgcaagcgcccttgcggaaacgtgaaacaccgcaggaaggagttccccggagattttatacatattcggaatcatcagcagcaggccctgggacgccgtaaacgtggacgtaagagcgcccgcagcgagcgaaccgtggacagcgccggcagcgccgccttcggactgcagctcggaaattttcatcgtctgtccgaagatattctttcttcctgccgcggcccattcgtcgcaggactccgccatcggcgaggaaggagtgatcgggtaaatcgcgccgacgtcggaaaatgcataggcgacgtgcgcggcagcggtattaccgtcgattgtttgttttgccattcttagtgacctcctaattttataattcatttaaaaacattcgcaacattataattatagtattacgggcactaaaaagtcaaggcagagcgccgctatatcccctttttttgtaattttggcagcatgctccgggggcgtataaattcatccgtatgtacgtacattttttattttagagaaagaggcgttttcttttccaaaaccatgccttgtactccatcctttcctgtgctatacttaaggccacaacaaaaaatgtgcagagtagattttgcgcgtcaagtgtatcaggatgggatgtcgcctgataacgaaaaggcccttgccttgcggtgcaaagtcgcgtccgctgctcacaaaagaagattctttgaagcggcttttgcttttttgcattttggccgacagcctttcttttgtagaagcgttgccatttttacgaaagggaggttatttttatgtccaaaatcagtcccaaaaccctgcgcctcgtgtattgcgcaacctttatcgccattgccgttgtagtcggcagttttctgtctttttacctgacggaaagtattaaattcaaccttgcgcccgtagttatcatgctgaccggcgcggtgctcggccccatttggggcgcggcggccggcggcgtgaccgacctgctgtccttcctcatcggcacggccgcaaaacccggtccttattttcccggctttacggtgacgatgatcctttacggcctgatatccggcctcatcttttaccggaaaaagcgcccgtccgagataccgtcgattgcaaaaatttccctcggcacggcgcttacgcagacggtttgttcgctgctcatcaattccttctggaattacctgctgtacgggccgacctacctcgtggtgctcactacgcgcctgccctccacatacattatgtgcgcagtgtatatcgtcctgatgtgcatcttgctgaaaaacaaacaaaagatgttcaaatccctttacgtgcccgttgcggcgtaagcaggaaaaaagcggcacatccgccgctttttttgtttccttttttggcagaggatttcgaattaaaacgtgaaaagccggttcctgagaaccggcttttcaaaaaataataaaagggtggggattgacatatcctagtctcctaagatacagtctcattttatactacatccggcaggattgcaagtgataatttgttaataatttatgaaccctgccttttcacttttcccgcgctttcccaagcctcctgagcacataattcggcagcgcgaagcagccccggtgaagctccgtattgtaataatccgtatggaggccaagcgcgttccacgccgccgcatcgaacacaagcggatcgcggtcctgggacgcgaacccgaacagccagtggccggacgcgtaggtgggcatatggaactgatagacccggcacacgggaaatacggaggaaagttttttatgcgcgcgctccatctcgaccgcgtcgccttcataatagggactttcgtgctggttgaccattacgccgcctggcgcgagcagcctcgcgcagtcttcataaaacgcctgcgaaaaaagcccttcccccacgccgaccgggtccgtggaatcgatcaggatcaggtcgtatttttcctgcgcctcccgcacaaaggcaatcccgtcgcagaagtgaagagaaacgcgcgggtccccgagcttcgacgcagtctgcggaaggtattcgcggcacaggtccaccaccatccggtcgatctccaccatatcgatatggcggacgcccggatagcgtacaagctcgcgcaccgcgcccccgtcgcccccaccaatgataagcacgttttttgcatctgcgttcaccgccatcggcaggtgcaccagcatttcatggtaaatgaattcatcccgctcggtcagcatgatgaaaccgtcgagcgtaaaaaaacgtccgtattcgtaggaatcgtaaaaatcgatcttttggaactcacttctcgtatcgatgaaatgatcctttattttaatgccaaatttcgcatttcccgtaaaatattcataaaaccagtcatttttcaaaatttctgccataaaaaatctccttcatctcccgctccaggttccgctcgatgcgcgcgcgcgtcttctccttcaggtccgccgcgtcgagattgaacaaataattgtcaagatcatatttcttcaaaaccatgctcgtgtggaacacgcgttcctcatacaggttgacgtcgtatgtgtcatatttttcgaggatatgcctggggataaaatcctggatggacataatatcgtgatccatgaacaccttgcctccgttttccgtgcgggtgaatccacgtacgcggtagtccatagtaataatatccgaatcaaagcttgtgattagcttctcgagcgttttcagcggcgatatttccccgcaagtggaaacgtcgatatccgcgcgaaaggtgcaaaccccgttctgcggatggctttccggataagtatgcaccgtcaggtggcttttatcaagatgtcccaccaggctcgcgggcacctcttcctccgcaatcagcatcgtgacagacgcgccctgcggttcgtaatcctgttccgcgatgttcagaacctgtgcgccgatgcggcgcgccacgtctgtcaggatctccgtcagacggcgcgcgttatagcattcgtcgatatattcgatataactttgccggtcctcttttgtgttggcataacaaatatcgtataaattgaagctcaatgtttttgtgagattattgaatccgtataatttcagtttttgcaaaccattcctccgcaccagaactgttttatttatagtttcattatatatcaacgaacgggaaatgggaaccatttatcccggcatggggatcacacttttgtcactctttcgtcgtgcttccttatgcaggacgtatatttgcctttcccggtcatctattataaatagtaccggataagaatgtattcgcagcccaaggaatacctgataaatcaagattggtctgccaatacagctaaataaggaggaacaacatgaagtttcaactcgcaacagattacgcaatacgcatcctgtgctacctctatgaaaacaacaacaggctttccaccgcgacccacctgtccgaaaagcttgggatcacatacctgtatttcatgaagctgacgggcgtattgaagcatgccggactgatccagtctgtccagggctgcaacggcggctaccagctcgccaaaaatgcggaggacgcgacgctttatgatgtggtgaaggtgatcgagggcggtattaagatcaaccgctgcctcgaggaagaccgtttttgcagcagggacgcggcggaatggtgcaccgtgcacacctttttccaggaacttcaggacggtttcatcgagcggcttgacagcgtccgcatccgcgacctgtgccaaaaaccggaaaagcagactgccgcccgtgaatacgcggccctcgaggtttaacggcaggcatccattataacgacaggaggaacattgtacctatgaaaaaaatacggcgactaaccacggctttgctcgtgattattttcctgttctccataaccgcgacggcgatggcggacggtgcgcccagtcttgttgatttaccagttacaggcttattgaacacagaatatacggaaacaacagatacggaagatacggaaacaacaacagatgcggaagaagacatgggtgccgggtctgaagcgcccggtgcggcagaggaatccacgacggagggcgcgtccgcaccggacggtgataatccttcgccttccccttctccctccgtcccggaggacggcacggcggcggaagccccggtgggaatttcgcttatgtcgcttattcccggcgcggacgttacggcaacaacctgggaggacctaaaggactccgtggagcaggcagaggtcgacggaaaaataatcgctctcgacccggcccttgcggggaccatgtccgccacggcagaactggtgatcggccacaataacagcgtggtaatcgacggcggcggcctcacgcttggcgcgccgtccggcggagcgcatttccgcattacaaattcgggcacgggtaccgttaccctgaaaaacatgacgcttgacggcaccggcggcacaggcagcgtcgtcctttcaggcggcggcgcgaagctcgataccgtaacggtcagcagcgtttccggtacggctgtctccgccaccggcacagacagcttaacgcttacaaacgtaagcgttgcaaacgcggccgcaggagttgcggcaagcgcaggcgcgcttaccatttccgattcctccttccaaacgatctcaggcacggccgtaacggcatccggcacggctacgcttgatactgtgacggtagacggagcaaaaaacggcgcggctggcgggagcggcctgctgaccatcgacggttcggaattcaggacggtcgccaccgccgccattacgcttggcagcggcggcgcggaggttacggataccatcattgacggtgtgacgaacggctccggcgtcgtgacgggggcgaatacactcaagcttacgggttctgcgatcacgaatgttacaggtacgggcatcgctggaaacagcggcaatattaccgtagacggctgcacattttcggaaaccaacggtcacggcatcagcgaaggaaaagacgtcaccgtcaaaaacacattgtttgaaaatgtgcgcaaccagcgcggcggccatggcagcgcaatctatgccggcacgaacctcacggtagaagcttcaagctttgtgaacagcgcctccacgctggacagcggctatattcagggagcgatcgtcggctacggaggcagcgggaaaacgatcaacgtaacgcgctcgtacttcaaggacaacaaggcctcccgctacggcggcgcgatcggcctttaccagtacggcggcactgtgaacgtcagctacagctattttgaaggcaacggcgtttccggcaaaagcgcaaacagcgacggcggtgcgatcggcgtctacaacaacagcagcagcgtactttgtacggtgaacgtggataacaatacgtttatcggcaatactgcgggcgacgacggcagcgcgctctttttcgagggccgcaacgacatggtggctgcaaacgtaacccataacacgttctatggcaataaatccaccaaatatttgctggcggccgcggacagcggcggcgtggtgcagctttccctcgatgtggtcggtacttttaccaataacacgttcatcggcaacactgctcccaattatgcaagcaagcatggggccggggcggcggtcggcgagcatatcgactccgcgaacgcggcggtgcgtcccacggccaccttcaaaaacaacattttcgtgggcaacggcggaaacggcggcaatagctacgcctcccgcaatatcgacgtcacggacggcaccgaccttggcggcaacgtaggctacgacaacggcacggctgttgcggcgaatatcacggcggagagcgtattcggcacttccccgcagccgcgcgcgaacaacacgcatcacggcgcggcgggaaaagcgggcagcgggtatacgggtatcctcacgaccgtcgtcatccaaccgaacgacggggtaacgggacaggcggataacacagccggcgcgcccgcttacggcacggatgcaagaggattcacgcgcggcgcctccaattccgatgccggcgcgctggagatcaaattcgtgaaattcgacgccaacggcggcgcgtggagcggactgcccggccttacctacgacggctccaaatattatgcggacgatacggcttccacgggttacttcctcgtaacggacccgggcggcagcgtgagcgtactcagcggcgaccttcccaccaatggtccgcttgtctttgacggatggtatacacaggcagacggcgggacaaaggccaccggcgatgtggccgcggccgaccagacgctgtatgcgcactggaaatccgcagcggcggaatacaccgtgacatacgtgggcaacggcgcagatgccggtacggtccctgccccggacaccgtgacggagaatacgctttaccaggtaaacgccaccccgcccacgcgcaccggctttacgttcggcggctggaaggcggacgatgcttcgggcacggtctaccagcccggcggctcgttcattatgccttcaagcgacgtaaccctgaccgccgtgtgggcgcccgttccggtcacatataccataacctatcacggcaacggaagcgacggcggcacggtccctgccccgggcacctataacagcggccagacagctgctatttccggccccggaacgatgtcccggacggaccacagcttcctcggctggaacaccgatccggctgggaacggtacgggatatacggaaggccaaggattcctgatctcaggaaaccttgacctgtacgctcaatggcaggataacgggcggccggccgtactctataccctgacttttgattcgcagggcggtacatacgtcggcccggtcacaaacatcgctgcgggaagcaaggttgcagcccccgcgccgcctgcacgcacagaatatacgtttgatgggtggcaccatactgcggactgtacatgcgcaagcgacgatccgtgctgggattttgaccgcgacacgataaatggccataaaacgctgtacgcccggtggatcccggcggcacaaacactatatacggtgacgtttgacgcacagggcggcagcggcgtcgccccggttacgggaatcgtacccggttcacggatcgcggagccagccgcgcccacgcgggacggctatgtgttccgcggttggtatcaggagagcgaatactccagcccgtgggtatttacggagcacacggtgaatggcaacctgacgctgtacgcaaaatgggaagcatcacctggcgccggtacggatgccggcgccgcggataccccaaacggcggcggtaaagtggaaagcaccgctaaaacaggcgatgcctccgccccattggcggtactgattcttgccgcggcggcggcagctgcggtgatcttccgcgcggtatcgacgctaaaaaaacgcaacagttaacagcctctgccctgttcctctccgggaacggcgcggaaaagccccggacagtgtgcccggggcttttcgtatatgtttcacattactaatccccctgccgtgcagcatccgccagggggattttttccgtttcaccggcagcgccttccgctccggacggtttggatgacagtaaaaaacggatgcgcttctagcacatccgttttagaatcccagtcagttattccccggccggtgccgaagactccgaaccgctttccgccgcttccgccgctgtcggcgcgtggccgaagcggtactcaatatccttgccgcggatctcggcgatctgcatcatctgcgtaaccgtcacaaatttatagccttcatccttcagcgccttgatgatcctgtcgtacgcatccacggttgtggcatggatatcgtgggacagaacgatagcgccgtcctgcacatatccgccatccggggtgccgttgaccacatggtcgtacacggtatccgggtcgcggttttccttcttccagtcgttcgggtccaccgaccacaaaatctgctcgcggccaatctgctccgccatgtcctcagacatagagccatacggcggacggtcgatcagcgcacgcaggcccgtcgccgcttcgatcgcatcgttggccttcccatactggttcgtcatgatttcatcaagcgaaagcttcatcaggtctttatgatcccagctgtgcgtgccgatctcgtgtccttcgtcatacacgcgcttcacaatatccggatactcggctgcgttggtcccgaccatgaaaaacgtcgcgaccgcatcgttttccttcaggatatcaagtagcttcggcgtgttttcgggatgcggaccgtcgtcgaaagtaagcgccactactttgtcatttaacggatcgatcccgtcaaggctcttcggggccatgtatccttcctttgcctgcagctgggcgaggctagtggttacctcgggcgtgggctcgggcgtcggttccggcgttgccgttggttcgggcgtcgccgccgcggacggttccccggtgggggtcgtggtaatcaccgtgggcgcggcggaagattccgccacgctgcccgtgccgccgcatcctgccgcaaacgcggaggccaccagcaccgcaagtatcatagagatcaatttcatttttttattcattcgttattcctttcagcggtcttgggggaaccgccgcttcaattataatacacatactgatatgttgcaaacaccagcggccagcgtgtatcaataaattcccggtatgcggctctttccgtcattcctccccaacagcttcacccgcagctggagcgctcctgaacaaatatgcgggcgtttctcctctgagctccgcgatctgcatcatttgcgagaccgtgacgaatttataacccttttccttgagcgcgggaataatacgcgcatacgcgtccaccgtggactggtaaatttcgtgggacaggatcacaacgccgtcctgcaccacgccgccgttccatccgttcattacatgctcatagatggcatccgcatccttatacttccaatcctccgggtccaccgaccaaaggacctgctcgcggccaatctcttccgcctgctcctccgtcaggttgccgcccggcgggcggtcgaaaagggtacgcaggcccgtcgccgcttcgatcgcgtcgttggccttcgtgtattgctccaactgctccgctgtggacaggcccttccattccgacgcttccggatgggtatagctgtgcgtagcgatctcgtgtccttcctcatattcccgcttcacaacgtctggatactcctcggcgaggttccccaccacaaaaaacgtcgcgactgcgccgttctccttcaggatgtccagcagctccggcgttttgaccggattcggcccatcgtcaaaagtgagggccaccaccttgtcgttcatcggatcgatcccgtcaaggctcttgggaagcataaagccctccttcgcctgcaattgctcaaggtccgttacttgcccgggagtgggctcgggcgttgccgccggttcgtcttcttcgggcatatgggatgcggcggcagtttgcccgctcgtaatcactgttggctctgcggcggccgccgcatcccccgcgccgcagcctgcaagcagagccatgcaggcaaggacggacagggtaatacagaccgttttcctcatcattttttatcctttcagcagtctctgaataccgttctttagattatagtgtacttgtaacaaagttgcaaactccttttccccggtaccggcgtatacaaaaagataaccgcagtatactgcggttatccggtcattttatcctgctgctttgcatcattcctcttcgccctgtccggcggaatcagccgcgggcgcactatagaacttaaagccgcccatatcctggccgcgggcttcggcaatctgcatcatctgggtgatcgtgacaaacttgtagccccggctcttgagttccttgatgatacggtcatacgcgtttacggtcgtctcatggatgtcatgcgacaggatgacgccgccgtcggaaaggtagccctccacttcctttgctccgccgccgtccccgccgttcatcacgttggaataaatgatatctggatcgcggtaatcggtcacccagtctttcgggtccatggaccagatgatctgctcccggccgatggacgcggcttcctcctttgtgatcgctccgcccggcggacggtcgattagggtgcgcaggcccgtcgccgcttcgatcgcgtcattggccctcgtgtattggtccaatttctctcctaccgataaattgggccattctttcgtatggttccagctgtgcgtgccgatctcatgtccttcgtcgtagacacgctttacgatatccggatatttctccgcattctgccccagcatgaagaacgtcgcaaccgcatcgttttcctttaggatatccagaagcttcggggtaagcgtctcgtgcgggccgtcgtcaaaggtcagggcgatcaccttgtcgcccatcggatcgatcccctcgatactttttgcggtcatgaagccgtcccgctgccggatctggtagatatccgtttcggctggcccggccggggccgtttcctgcgcagttccggctgcggctgcctgctccgccccgaggaccgcttccttgttcccggcattcagtatgcccgcaagccttgcaagcgggactcccacctcaaagcttcccgccccggaaggcgcgatcgtcccgtcgccgaagaaaaacagcactttatccggcgctaacacgaaattcgcataattggcttcctcgggagccgttccctgtgtgaacaggctttcatccatatttttcagcagtacttcattcctgcgcagatattcctgcgcagcctgcgcaacctgcgtgaggtaatcctgtgacggatcgaacacatccgaaagcgcgagctttttgcctgccgcggggtcgtacacaaacgcatcgacttcctccgccgcagcctgccccgccagctccgattccgtcgtaaactttatactgagcaggttcccttccgtaacatacggcttataggtcatcgtcaggctgcacagcccatctttgttttccccgtgcgcagcgtccgcagacgcgcggaattctgcgagcttattttccgcaaatgctttcagctctgccgagatatccggcgcgttcgcgaccaccggataatcgacggataccagggtatctccttccgtttccaaaatatgtccgctcgttatcccttccgccgaatcaagctgcctgctcaccgccgccgcttcaaaccgtccccggtaggcttccatgtccccggttccatttcccgcgcaacccgccgcaagcggaagcgccaacgctgccgccgcaaaaagcgcgatccatttccgtttcatactttttcccccgcgccggacgcccaatacccggcctttgttcctatgcttgccgctcccagacctgatccgcggttttgaagcgtctttcgccaccaaaattattttcccatattataccacagaacctatatttatcacaatttgtttccgcatgccgcgtgtaaccttccacgacggagtttgtaatccctttcccaatatgatacgatactaggtacgagcaggcgggtgcgttatggattactcaaaaaagagcctttgggcttttttgccaaacaggaaaagcgaaacgaaaccagtggatgtcttttacgtatatcccactatttacgcgcacccattccagaaaaaacgccatcatatgagcatgaggaatccgatctacctgtgggtcgccaaggggatgtctgcctggcagggccagctttttgcgcgccattgcaatttttacgcgccgcattaccggcagcttggcacagaaagcttcaaaatgcccctgcctgaagtcatgcgcgcggaacggatgccttatgaagacgtacgcgatgcattctactattaccttgagcattataacaacggacggccgttcatccttgccggacacagccaggggagcgccatgctgctccagctcatgcgccgggaattttccgatccgcggctgcagaaaaagctcgtcgccgcctatttgatcggcttctcgcttacccggcgcgatttcaagcgttatccgcatctccgccttgccggggcggcggacgatacgggggtcattatttcttataacacgaccgcgcacggccttcccctgatgcggtttatccgcccggattcgctttgtgtcaacccgctgaactggcggcacgacagtgtatatgcggataaatccaagaacgacggcgcggtgctgttccaattcgggaaaaggttcaaatacgaggtgccccactatacgggcgcgtatgtggatgaggcgcgcggcgtgctgatgatcgacgacgatgcggcttacgagctttacaaggcgcggtggttcttcaaaaaatttctgatgaaccgtggatcgctgcacatgctcgatatcgcgcttttctataaaaacctcgagcgaaatgtgggggaacgaatcgccgcatatttgtcccggccttccgaatgacggcgctgcgcgcccccgcaggataaatccgcagtaaaaaaaccacggatactcaatccgtggtttttagctctttcggctttgtgatgatgcccgcgaacagttccgcctcgccattttcgtcccgcgatacccggccccggcaatggatccacacccattttccgtccttatcgcgcacccggtattccaaatcatgcgtatcggtggccccgtcgcgtatttcctccatcgattgtgcaaaggcagggcgttcgtcctcatggatcagcatgctccatatggaatctccgccatacacaacctctcccggaagcgcaaattcttccaccattttcggcggaaaacggaacatattcgtcttcatattgcagatatagatataatcgtccgtactggccgaaagcgcctcgtagaggtgctccttatcgtaggaaaaattccccggcatgcccttttccgcccgctcacgtcccatcctgccgttgtggtatatccgcttctgccggtacatccgttcgtccacttccgtggtgatcttggttatatcttccatgtcctgcgggccgacttccagcactccatagcaaaagctgatctcatacggctttttttcccgttcccggatccttgccagttcctcgagcgcatgctgcatgaccctatccgccacccgttctccgcagccgtaaaacacgaccataaattcgtccccgctcatacggaaggcgaactggcctttttccagcgaacccgtaaccaccttcgcaatcgtggtgagcaaattatctccctctacatgtccgtaggtatcgtttaccgttttcaacaggttcacatcatagaggcacaccgtaaccgccgcgctttcttttttggcccgcgcaagcacttccgaaagccgctccttgcctgcgcggcggcccatcatccctgtgagctcgtcgaggctggcctcccgcgacatcttttttgcgctcgtaatatccacggattgctggaaatgcacgatgcgcccgtccagccagcgcatcgtgctgtcataattttcataatgcctgtgcgtgagcgtattgtattcctcccacaggtaggatggcttttccgccggattgttccgcagctggtcgatcgggcagaaatcacaacgctgcgtcttatccgcctgcaaaatcttccagcatattttaccgataggttcctccacgccaaactctttgcgcatggtcttattcataaaaagaatttcgtccgtatcgagatccgttacatagatattcgcccgcatattatccatcaactcgttaaacacccacgtctgctccatctggttctggcaatagatccggttggcaagggcgatcctcgaggtctgcgcgagcatttccaacagccttttttcctcttccgtccattcctgttctttttccgccatgattacgccctggatgcggctggaataagaaacgggaaccaccaacaggggcgcgtcttcttccgtccggcagtccaatttcatgatcctgtcaagcttctcatcgtctgcggcctcgtccgtccgggcaaaatccttccattcactgaaatccagatcaaaaaaatgacggaccgcctcctccggcaggatccttccattgcgccacaccgcggcttcacggacccggtagtcttcggagaggccaatgagccccacgccgcagacaggcaaaaaacgccggataaaaacggcaaagctttcgatgatttccgcaagcgggccctgcccgatcacaaagggttccagttcttcgagcgcttcattcaggatgtttaaatattctgttttcccacagttgccgctcattaaaaaatacccttccacgctagtatgttttaaagatattgtgtttattatacgccttcgctatatgaaaaacaatcatatttcttattttaacaaaatccatcccgcccgctgaaacttccgctttttatcactgcttgggcgttccgtccagtcatttttaattgtaattgtttttcaattacattattgtacattataacgcttcctcaaataaattacaatattaattacaggagaagcagcggtatggaaaaattcaaggtcaatacaaacgacggcaaaatttcgtccatcaaccggacgatccgtttgaagccggaatattttgaaaaaattatggagttgagtgaaaagaccggcgtttccttcaacaaaatcgtgaaccagtgtattgagtatgccttaaataatatggaagaaaaataatgcagatatcaaaaaagacgttgccggagcagcgtcttttttgatgtttccatttactgaagaatgccgtactgtacgataaccgccgcaaaaacgaccgccaccatagacatcagcttaatcaggatattgagggacggaccggacgtatccttgaaaggatcgcctaccgtatcgccgacaacagcggccttgtgggcttcactgccttttccgccgtgcgcaccgccctcgatgtatttcttcgcattatcccatgcgccgccggagttggacatcataaccgcaagcacaaagcccgtagcggtcgcgccgccgagcataccgactacgccgttcgcgccgaggataaggcccacgatgatcggggcaacgaatccgaggatcgccggagcgagcatcaggtgcagcgcgcccttcgtgcagatgtccacgcacgattcgtaatcaggctctccttcgcctgtcatgattcccttaatctccctgaactgccgccgcacttcgacaacgatcttcatcgccgccttcccgaccgcgctcattgtcatggacgagaacaagaacggaagcatcgcgccgatcagcaggcccatcaggaccgtcgggtcgagcagggagagcgccgacatatcgatctggcactggtccgcaaacgctgcgatcaacgcaagcgccgtaagcgccgcactgccgatggcaaagcccttgcccgttgcagccgtcgtatttccaagcgaatcgagcgcatccgtacgttcacgtaccaccggatcctgaccgctcatttccgcgaggccgcccgcattatctgcaaccgggccgtaagcgtccgtcgcaagcgtgatgccgagcgtggaaagcatgcctaccgcagagatcgcaacgccgtaaagaccgacttcaaagctgccgctgccaagcgttccgcccgaagcaaaatagctgatgagcacgctcgcaaccactacgatgaccggaaccgccgtggatttcatgccaagagcaatcccgctgataattgtggtcgccgggcccgttaccgtagactccgcaaggtctttggtgggcttgaagtggtcggacgtgacgtattccgtatagtagccgatgataatgccggcaacaagaccggaaaggatggccacatacacgccgacatggtccggcccgaggattcccgttaccaggaaataacccgctacggcaacgatcgccgctgcaatatacacgcccttatgaagcgcggaaagcaggttcttctgcgaagcgttctcctttgttttcacaaaaaagctgccgatcacgctggcaacgatgccaagcgccgcgagaaccatcggaagcgccagtccgccgccgccgaggcccgccgcaaccgcgagggcgcccgccgaaataatggacccgacgtaggattcgtaaaggtccgcgcccattcccgcaacgtcgccgacattatcgccgacattatccgcaatggttgccgggttgcgcggatcgtcttccgggatgcccgcctccaccttgccgacaaggtctgcgcctacgtcagccgctttggtgaagataccgccgcccacacgcgcgaacagcgccatgcacgaagcgcccatgccgaacatcagcatgttgctcgcgatctgcgccgtatcgcccgcaaaatacgcgtttagtataaaataccagatcgtgagatacagaaggccgaggcctacgaccgtcatccccataaccgcgcccgaagagaacgaaacgcgcagcgccgaattgaggctcttcgtcgcgcccgtgaccgtacgggaattcgccgccgtggcgattttcataccgatgaaacccgcgagcgcggagaatacgccgcccacgatgaatgcgatcggtacgaagaaggtcatgaagcctgttgcggccaagatcgccaggacgatggttacgatgacgaaaaagatcgcaaccgtcttatactggcgtttcaggtaggcgttcgcgccccgcctgatggcaccggcaattttcttgatcttttcgctgccttcgctttttttcagcacaccggatgcagcgaccgctgcgaacacaagtgcaatgacagcgccgatcaaggcccaaaacataatgtccattggtacttcctccttatgattgcatatatttgtagaaactcttatacaggcgcgcccgcaccgcccaaaccggatatggcaaaagcgcgtcatctatttcaaatatggaaatcggccttccgcgtcctgccggagcaaggccgcctttgcttgcttcttcttgttgctgaaaagttctacccgctgcccgcgcggcttttccgccgggtttgcacggagtcccgggctgtctgtcttgatgaaaacccgtaccgggagcggcaccgtaaaagtagttcgaaaacctgaccagcttcattataatactttttttgtttatttgaaagtaaaattggcaatttattcccgctttttttgaacaatttcaatgaaattctcaattttttcgaacattgctgaaaaatatgcacgatttcccattttgacgggatttgacaggtggatttggataaattataattgaattgtactaatacacttaatacagagataggagacgctgccgcgctatgatggtaaagcttgacccggcatccaaaaagccgatctatgaacaaatcgtccaggaggtaaagtcgcttgtcatgacgggaaagctgctgccgcatgagcaggtgctttctgtgcgcaaggcggcaaaggagctgggcgtcaatcccaatacggtccaaaaatcatacgcgatcctcgagcgggaaggcatcctttactctgtgacgggaaagggcgattttgtggcggacaacgccgcccgcatcaaggagatgaaaaagcaggaaatccgtgatatgttcatacaggtcacgcgggaggcgcgcgattccggtttgtggatcgatgaaatattcaccatcgtggacgaggcctactcgacaaactgagaacgggttttccgctcttgcgccgttccgcacgatcagacgtccacagccgcccgaagggcggccttttttatccccggccccggaaacccacgttcccggcggccttattttaattttcccggcggttttggagtcttgcggaaagcgtataaaataattatcaaacggaaaggacaaacagaaaatgctcgatgcggttcgccgtacaacatgcggaaaaatatgcgggacggtggaaaaggacgtccatgtatggcggggcattccttatgcgcagccgcccataggtccgctgcggtttcgccccgcgcagcctgtctccgcatgggacggggtttatatggcaacggcattcggacccgcctgcccccagcgcaaaagacgcgtcgggacgagcgaggactgtctctacctcaacatctggtctccccggccggacggtaagaagcgcgcggtgcttttctttatccacggcggttcgttcgcgggcggggcgggcagcgatcccgaatacgacgggacgtccctggccaaaagcggggacgtagtggtcgtgacggtcaattaccggctgggcgtgctcggttttctcgatttctctttcctaggggaagatttctcgccgaactgcgggctttccgatatcgttgccgcgctcgcatgggtgcacgggaatatcgaagcattcggcggagacccggaaaatatcacggtgttcggccagtctgccggggcgatcgccgcctccgtactgcccgtaatgccttctgcgcggaactatgtatctaaagttatcatgatgagcggcggaccgtcgcttttgtatacgaaggagcattaccgggaaaccgcacggaaattcctttcatttatgaatatctccacgccggaggaactgcgcctgatccccgcggagaagctggcggcaaaacagaaaaaatttgcttcctgctgcggcctcggcgacggaacctttatgatcgaggtagacggagcgctggttcccgattacccgattcccgcggcggcgcagggcgccgcgagggatatccctatcctgatcggcacgacccgggaggagctttccttttcctgcaaaaagcttctttccagggtgctcgatatcggaagcatcttaaaatcagaccgcaacagagaaagcgcagagacccggcagcggatcagggaggcctacagacggtatggacgccgtgcggaagccatgatgctgacagaccgtgttttccgtatggcaagcgtatggtatgccgaggtctacagccaatatgcgcgcacatggatgtaccgtttcgattatgaaaccccggccatgcagcttaccgccctgcatgcctgccattcttcggatatcccgtacgtcttcggcaatctccatgcccggcagtataaatggatgttccttctgcccgcgccgcgttcacgccgcaaagtgctcgcggagattcagggggatttcatccgctttgccaaaaccgggcttctcccgtggccgcggtgcggcagcggtatgaccccggcaaagtgctatgacaaacgctgccgcatcctgcccatggtggagccggagatcaagcgcgaatatgaaaaaagcgaattcaagcgccacagctttaaagggcttgactatacgggagccggtcagcgataatagagggaaacaagctgaagatcggaggatgccgtatgacagatctatcggaagtccggaagctccttgcggaatattctacgtgcgagataagcgaaattacgccggaaaagcgtttaacaagcgacctggagcttgattccttttccctgcttgacgccgttgccgcctgtgaagaccacttccgcctttctatcccggacgaggatttaaggcgcctcgataccgtccaggatgtggtggactatatcaacgcgaaagcaaaatagcgaaaagagcctcaaaagccgtattgcgtctcatgcgcaggtacaatctttcctgggcaggtttcgccgcccaaggccattctgtcagcggcagcaacattttaaaacttatacaaataagtttagccgtgatttcagggctgacaaccctaaccagaaacggacgacggacatcatctgcatacatacgaaagaggtttgatccgctttcccctattttttctttctcgagaggttccagatggaaagcgcgtaaaacgcagccgccgtaattaccatgaccgcaatacatgccgcgggcgtcgtcatttccccgccgaattcaaatttcacgccaagcgccgtatcgaagtcccgcaccatgctttcgggcgcgccggcaaaggaaaccgcggcaggttgctctgtcatcactgtgcggaaaaaagacgcggagtaagagaccggaaacgcccggatgatcccctgcaccgcctcgggcagactgccgatgggaatatagacgccggtaagaaagccgatcagcgtgccgatgacagtgctcgccgttccgaacgcattctggcttttgaaaaacgagacgataaaaagcacgatggagctcgacgacaggaccgaaagaaaaatcccgccgatcaccttgagcatattctccatggaaagcacctccccgccggacgcaacgataaaaatctcgccggcaaccagcgcaagcaggctcatgatgacgcccacggcaaacgcgcagaaaatataaccgcccgcgaggctgccccgtccgatgggggacaccgtaaaatccttgacgatctttttcgcgcggtcctccaccatgatgccgaacgcgcccagcgtggacgtcatggagatcactgcgaggatccccgcgatgatccagctgttcatcagaaagtcgatcccttccacatcttccatgccgcctgtaatcgtgtcgccgaggaacaccgcatacagcgcgatgatgataaatacggcgagcagggaaaaaaacaccgagcttctgtccctgaagaatacttttaagttgcgttttgtcattgcccacataatcagttcctcacttccgcgcctgtaatgttcaggaaaacgtcgtccatcgttccccgccgtacctcgaatccggcaatctcttcccggcatttatcgataatgccgattgcctccgctgtgtgccgcagccggatgataaaatctccgcctgttttttggaaagcgatcccttcccgcttcaggaggccggaaagcttctcttcccgtttgggcgtgatcttaagcatatcccgggtataattttccttgagctccgcgggcgttcccttggcaagaatgcgtccgcggtccatgatggtgatgtagtccgcctgcgccgcctcttccatgtagtgcgtggtaaggaagattgtcatgccgcgctccctttgcagcaggcggatcgtatcccataccttgcggcgggtctgcggatcaagacccgttgtcggttcgtcaaggaatagtatttgcggcgtattaacaagagcgcgggcgatatccgcacgccgccgctgcccgccggaaagctttccgtacgggcggttccaaaaccccatcacatccgctgtaaacgccgcggatgtgaccgcgtccctgagtgcgtcttttcccattccatagaggcttccccgcagcagcaggttttcgcgcacggtaagcagtgcgtcaagaacgctgtcctggaaaacgatgccgatttccgcccggatggccccgtcttcccggcctagctcgcggtcgtccaccaggacccgccccgcatccggcgcgagctgcgtagatattatgtcgatcgtcgtcgattttcccgccccattcggccctaaaaacgcaaacagcgctcctttttccacataaaagtcgattccattgacagcccgtacatttccataggattttttcaggccttccaccgtgataatttttgtcatgcttctcccgtcctttcggtttttacagggtttacgttatcaataaaaaaagaccgcttcaagcggttttgcgcaaggtgcattttttctatgctaaagtgcagaaaagccgcccggctacagggacaggtggaacaggcaggacaccgggaaaacaaggataagcgcgggcagcatattaacagccttgacgtccttgattttggcgatggcaaagccaagcgcaagtgtcatcagcccgccgacggcaataaaatcggccaacatcggttcgcttataaggggcatgataaaccggctggcgtaaaagcaggcggtaaggatcaaaaactgcgggaggacaatgataaggatcgcagagccgagcgccgtggcaaagataagggacgcgaaaatatccatcgcggccttggaaagaagcaccgtgaaatccccggtcatcccctcggtgaaagcgccgaagatattggtgccgctggcgcagaagaccgtcgccacaaccacgtaaaaattcatgtactccttccggtccccttcgatcttaaaatgcatgcgggagatcgctttcccaaacagggcgcggacccggccgtccagccgcagtaattcccccgccaacgttcctaggataagcgacaggatcacggccggaagggaagacagctttgtcagggagatcaccccgatggcaatcgcgcaaatcccgaaaacagtattaaggggctgcgaaatgcgctggggaaccttgtcctttattttagccccggcgagcccgcccgcgacgatacacaggcaatcgattattacccctaccggcatgaatgtttcctccctcgtatggtttcaaagacggtgtaatataacataccgggctttggaagtcaagcggaagtacaaataaaaaggcgggcttccgcccgccggaccggcagctccggtcatttccgtttcatttttttgatctgcttattgatatcctccgcgtccgctttgtttttgataacaagcacaccaaaggtcgcgaggtatacgacgccgatcatcccgagaacaattggcacgtagggcatttccatgggaataaagcccgtgggcccgccgatcaggaacaccaccgcgacaatatcggccaggtcgacaatcatctgcagcgccgggctgctgatcggcagcctgtcggtaaaagaaatcatcaccgcgatacacgtcgtcatcaggaacaggaccagcaccgcttccggcaccacggccgcaaaccccaccgtaaccgagagtatcgtaaacaaaagcatcagtacggtaaaggaaacgcaattcatggtaacgtaaaacttaaatttcgacatcttccttttcctccttacaggccaagcttttgcttgagcgtttctacataatggcgtgtgacgatgactttttcgccattctcaagctccgcctccatttttccgttcagcagcgcccgcacatttttgagcgtcattacgttcaggatgcacgatttgctgatgcgcacaaaattagcctttgcgacctggctttccagctcgtataatttcaggccgcattcatagacctcttgttccgtataaacgaaggtgcggttatccaccgactcaaaatagaatacatccccgagccgtatctggaaggtctttccgtcccgtttggctatgacggaaaaggaatacagcctgatctgctcgatcagtttttcaagtccgggatcgataagcccgcatttgatggtgacctccacctcttcattgtccggcgactggtcgatcagaagtttcaacccattccctcctcgttacggcctgtacggcgcaccgtcccgcattcgcctgcggggcatgcgtcccttttgctgctccttgcgcggccttccgtcaattggtaaaaaatccaagctgcctgcccgccggttttttgcgcgtatgcgcggggccttcggcaagaagtccggaaggaatatccgcaagaaaaggcgagcgttcttcttgccttgtgagcataacaagctcgtcctgtgcgcgggtcatgcccacataaaacagcctgcgttcctctccttcgctgcattccccatccttcctgcggaagggaatcaggccgtccgttacgccgcacaggaatacggccgggaattcgaggcctttggccgcgtgcagtgtcatcagcgtgaccgcgtccgcggtatatgccttctgcccgctgcgctttaggtcgccttccgttccgagcgtcagggcggttagaaagtctgccatgcggtgatggaacaccgccatgttcctcaggcgtttcaggcacgcgggcgcttcttccccatatccttgccctgaaatccatagatcgagaagctccgcaggagcagccttgcgcatcagcggcgcgcaggtttcaaaatgtccggcaagctttgttccccccgcatcctccaaaacagatacaagcgaagctgccgtttgcttcctccgtccgtaagcgtccaatattttttccgccactgccggggcgacgctgtccgcccgcaggcaggtcccgagggaaacgctgtccttcgggtcgcacaggatgcggaaaaacgcgagcgcgccgcgcacgcggcggtcagtgagcgtattatcctttcccagcaccacataggggattccctcttttacgaggcattcctcgatcagctccgcctgccggtgcgtgcggtaaagcacggcaatatcggacaggccataagcggtgtccgcgctttttttatgctgcgcatgggcggcaagcatgtcgaccccgcccaccatagcgttaatttccttcgcaataaacagcgcctccgagaagggagagtccgccgctaaaagccgcgtttttacaccgctttcccgctgcgcttccagcacaggttcactgcggcggagcaccgggagggcgcagccgatgatttcgggcgtagaacggtagttctttttcaggcgtatttcacgtacaggccggtcctctttgaggcgctcaaagcaatgcgcgtccgaaccgcggaagccgtaaatagattggtccgggtcgccgatcacaaaaattcctttgctctttttcccccatgcgcggatcaattcgagctgtacgggattgatatcctgaaattcgtccacaagcaaatgctcaaagcgcggacagagcgtttcgcactttgcgccgccagcacggaatttctccagcgtatcggccaggatatcgtcgaagtcgagcacgccgagcgagcgcagccgagcgcagtaagcgtcgtacaccacctggggaatatcgcccgccgcgccgttcttgattttagaaaccgcctgtattgcgtcgcggggtttcatgacacagttacacgcggtaagcgtttcggaaagaaccgcctgcgcttcctgtccgtcgatcaccgttatatttttatcccattctttcagcaggctgagcccaagggagtggaacgtgccgatgtgcattgcccgcaccgcctttttatcgccgaattgcgtctccagcctctcccgcatttcgcttgcggctttattcgtaaaggtgacggccgtgatcgcttccggcgctacgccgcactgctcgacaagccatgcgatgcggtacacaagcgtacgtgttttgcccgtacccggccccgccagcacggcgaccgtgcgttccggggcagatgcggctgcccattgttcttcattgaggccgtaagggtttccgcccgtttccacgggtgccgcctcttttgggctttcgtccgccttaggcggtgccgcctgcgcggacgcatgtttttccggtttctttttgggttcgttcgcaagcagggaaagctggccggacagcgcctgcacctcgcttttatccatcacgagaatctttccgtattcgccgtcgtagcccggatggatttccacctcgccgcagcgcaggcggcggattccctccgcgatgcacacgcctgccgcccgttcgatttccgcgagcggcgttttgcgcaggatggtaagctccggaccgagcgtcccgataagctccagatattgcgcgcgcaccttcttgctcgcaggcgtaagccccatcgacgaagctaccacctcgggcagcggcacgatgctttcaaaatgctttgcattttccggccgaaatccttccgcgcggtccgcaagctccaccacacggtggtagacgcccacggtgatgcgccgcccgcagaccgggcatttgccgcccgcggcggccgtctcctccggcgtcatgcacaccttgcagttccggtgtccgtcgttatggtatttcccttcctccgggaaaaactcgagcgtcccgtaaaattcatccgtatcatggttttcgagcgccgccgcaatggcgggatagctcatttccgtattgaagagattggcttcccgcgcaaggttcgcgggagaatgcgcatccgaattggataccatcgtgaatccgtcgagcgcagacagccgccagttcataggcggatcggacgacaggcccgtttcgagcgcatggatatgcggcgtcaggtctctaaagcattcttcgacggcgtcaaagcccgaataggctccgaacagcgaaaaatgtggcgtccaaatatgcgccgggataaagactgcctccggacagatatcaagcgtgatctcaagcaggtcccggctcggaaggccgaggatgggccgtccgtccgaatgcaggttgaacgagagctcgagcttccccgccagcgcctctgccgcctcgagcgagggcagcaggatcacattgtggattttgcgtaccttgccgtcctgtttatagatagagctgatctcccccgaaaggataaaccgcggcgatgcgggattgcctcccacctcatccgcaaggcggtattcctcttttaagcggtacaggccttcttcggcaggagccagcttttgcttcagttcctcccgccatgcgggatgcgtaaagtcacccgttcccagaaggtcgattcccttacggcgcgcccacaggtcgagatgctcaggcacgcattccctgctggtcgcacgggaatatttggagtggatatgcaggtctgctataaacatttaatattccgttccttttctcggttcaaaatgatcgcatttgttcctctcgcccgtcagcatatcgggatcgatatccccgtacacctggcaggaccgtatttccggcatgccgggcgcaaaggtcagcacgccgtagtgcacgcagttatagcacagcacgttttcctcgttgacataattctgctttaattggattcccaactgaattctctcctttatgattacatgttcgttatttttcatacccgtggtctttggaaaggtatacagctttttatagaagccgccggcaagataaaccggcacgagcgggttatgccccggaaaggccgctttccgctccaaaggaagggcagctctccggcgcatgctccgcatcgtgcctgaggcagtagcccttcgcgcagtccgcacaaaaataagaagacataggcggttccccgtttccatagaataactttattataccacgaaaaaagcggcggtgaacgctcccgccgcttaaaatatccgtaattacagcaattcgccgtactttcggataaacattttcttgagcgccgtcacgagcgccatatatgcgattaccgtaacgaacagcagcaggaaaaagctgcctggcatcgccatcatcccaagctcgcgccccacgatcgtgtagggaatgaccgtgcccacggcaatcccgatggatgtgagcagcgaaagctgccacgaggcgcggctccttataaacggtgtatgcggcgtgcggatcatgtggatgacgagcgtctgcgtccacagcgattccacaaaccaacccgcatggaacagcgcgacaaagccctcctgcgccgtttcgccaagcgtgtggtacgccccgccaaacaccatcgggcacagcaggaaatacattgcgaggtacgtcgtgatgtcgaacaccgagctcgtcggcccaatccataccatgaatttgctgatggaggaagcgtcccacgcgcggggcacgcgaaggtaatcctcgtccacattgtcccacggaacggcaaggcacgaaacgtcgtaaatcagattcagcaggagaatctgcacgggcatcatgggaaggaagggcagaaaagcgctcgccgccagcacggaaaacatgttcccaaagttggagctcgccgtcatcttaatatacttaatgatgttggcatacgttttgcgtccttcgatcacgccctgcccgagcaccgtcagatccttttcgagcaggataatgtctgcggattccttggcaacgtctaccgccgtatccaccgaaatgcccacgtccgcctccttcatggcgaccgcgtcgttgatgccgtcgccaaggaagcctaccgcgtgccccgaggcgcgcagagcacgcacgatccgcgccttttgctgtggggaaagcttggcgaacacatgcgtccgttccgccgccgcttcaagcgccgcgtcgtccatttcttccacgtccgcgccgagcaggacgtgctttgccgggatgcccacttggcggcagatatagcgtgtgaccgcatcgttatctcctgtcagcaccttcacgtccacgccgtaatctcgcagcatctcgagcgcccgcgctgtgctttccttgggcgggtcgaggaaagcaaggtagccaatcagcaccatatccgtttcatcatccacagagaacgcgccgcccgtatcctcgctcagtctatgcgcgattcccaagacccgcatgccatccgcgttatatccggcgacggtctcaagtatctcccgccgcacctcttcggtaagcgggatcacttcccctttgtattccgcatatgcgctcaccgcgagcatctcttcgatagcgcccttggtgatgatctgtgtttttccctttttatcagcgacaaccacgctcatgcggcggcggttgaaatcaaaaggaacctcgtccaccttgcggtaatcctccgcgagttccgccatccccacatcctgcgcatggttcatgacggcgacgtccatcaggttccgcaggcctgtctggtgatagctgttgagaaacgcgtggcgcagcacgcgcgcatcttttttgccatggatatcgagggaatattcgagcaccaccttgtcctgcgtgagcgttcccgttttatccgtgcacagcacatccatcgcgccgaaattctggatcgcacccagctgcttgacgatgaccttttccttcgacattgccaccgcgccccgcgcgaggtttgccgaaacgatcatgggcagcatctcgggcgtgaggcccacggccacggaaatggcgaagagaaacgcctgcatccagtcgcctttggtaaaaccgttcagaaacaacacgatgggcaccatcaccagcataaaacggatgagcacccacgaaacggagttcacgcccttttcaaagctggtcttgggtttgcgcgccgtcacctgccgcgcgagggaaccaagcaccgtatcgtcgcctacggcgatcacgatggccgccgccgacccgctgaccacgttgctgcccataaaggcgaggttgtcccgcccgagcacgtccggcccgcccgagcgcaccgtgcgtccaaatttttccaccggctcgctttcgcccgtgagggaggattggctcacaaacaggtctttcgcctgcagcacccgcaggtcggcgggaatcatatcgcccgccgcgaggcgcacgatatccccaacgacaatttcgtcaagcggaatttcctgttccatgcccccgcgcaccacggcggcagttgtctctaccagttctgaaagcctttccgccgcccggcccgagcgtgtttcctgcacaaactggagtgttcccgataccgcaaccatcgcaagcacgataataacggcaagcgcatcctgttcgcccggctcggcaagcactacgtccgtcacaaacgagatgacggcgagcaccagcagcacgatcgtaaacggatttaaaaacgccttgacaagccgttttaaaagggaatcgcctccgccgtaggtgatgcggttttccccgaagcgtccgcgcatatcccgtaccgcttcctgcgcatatcccccgtacgtattggaataccgtttcaaaacgctgtcctggcttttgcctgaagcgaggagcagcctttcttttacttcctgcgccctgcacgtttgcagggcaacgtttacttttttgttcattggtccttacctcctgtttgaagttccggacccggagaaacgagacgcagtgcgccgccctcctgggccctggtctggtttttggtccgccatgtccacgtttctcacttcctttcgcaatcaaatcatagaaacaaaaaatcccgccgaagcatgaacgcaacagcgggactgttcccacagaaacacacgaaaaaacgcgctaccgttcaagcttcagcatcgcagggcaatgaaattgcattagtctatgccttggtttcgacataaactgctgaccctctcatagtgtctccactattttgcggcagcaatccgtatccctgcggtaacctcgcctaccgaaatcgaactattcatttccggtttccagtatacccgcgcgccgcgccgttgtcaactttagtcccgataataccggaggattacgcttgcaggcacagcacgtttgcggtaagatatccatatatcaattaaccacaacttgcggaggcggaacatgaaatttgcgaataaaatgaaagtgttcgatttgaatattttttccaaactggaggaaaagcgcgcggaactcctccggcggggcatggaggtcatcaacctttcggtgggtacgcccgatttcaggccggatgaccacgtaatcgcggcgctgaaagaagcggcgggcatagcgggtaacttcaaatattcccttgccgatatccccgaactgatccaggcggtgatcgcgtggtacggccgccgctacggcgtttcccttgagccgcggcaggtgatgtccgtctatggtacgcaggagggcatggcgcatgtagcgctcccgatctgcaatcccggcgaccttgtgatcgcgcccgacccgtgctatccggtgtttatgtttggcccgcggatggcggaggcggaaatttattatacgccgctaaaaaaggaaaataattttctgattgattttgacagcatccccgaagatgttgcccggcgggcaaagatgatcgtcgtttcttatcccaataacccggtgacggcggtcgcggattacgccttttatgaaaagctcgtctcatgggcaaaaaagtatggcgtgatcgtgatacacgacaacgcatattccgagcttgtgatggacagcgaaccggggatgtcgttccttgccgttccgggagcgatggatgtgggaatcgagtttaattccctctccaaatcctacaacctgaccgggctgcggatctcttttgctatgggcaatacggaaatcatccgcaacttccgctgcctgcgttcacagatcgattatggcatttctttcccggtgcaaaaggcggctattgcggcattaaacgggccccaggatattcttgaacgcaaccgcaagggataccgcgagcgcagggacgcgctgtgcggcggcctgcggagcatcggctgggacgttcccgattccccgggtaccatgttcgtatgggcgccgctgccaaagggctatgaaaagtcgtgggagttcgcgcttgagctaatggataaaacgggagtgatctgcacgcccggcgaaagctttggccctgctggcgaagggttcgtgcggcttgcactggtggcagatgtgcccaccatcgagcgtgcggtgtgcctgatccgtgaaagcggaatgatccgatgagttatggatggctcggcggttatttgctccagaaagcgggcgtgaccacagactaccaacccgaatggcaagccacccgttatttcgtaggcggaaaaatgtttgccatgcaaggcggcgacaggaacaaaagacctatttttacgatgaaactcgagccttcgctgggggatttcctgcgaaggcagtatcccggcgcggtcgttcccggctattatatgaataaggtgcattggaattcgctgtaccttgaaagccgcgtccccgaagaaactgtgcgcgaaatggcagataaagcgtatgaagccggcctttgcgcattatcgaaaaaaaaacggagcgagatcgaaaacgggaaggaatagaatatgctgatcgattttgaacaactggacgaaacggagatcaaaaacttctatggcggcacgcaatccacttatgcgcggatgtttgcggacgaaagcaaccgtatcatgcgcggacggcttgcgcccggcgcgtcgatcggtccgcacacccatgaaacgggcagtgaaattattttcatcctttcgggaacaggaaaggcgttttgcggcggagccacggaagccctgacgcccggtatgtgccactattgcccgaagggacatacgcacagccttacggcggacggcgccgaggcgttggtcttttttgcagtcgtcccgcagcaataacaagcaaaagaggcgcttccgcgcctctttttttgtcttttttccgcgtgtgaatccgttatgccggcatcaggctgccgccgcccgcgagggcgattgtctgtgcgccgagccattcgtttcctgccgcgtccgcatcgtaatacaggttatgccctccgctcacaatatttgcgcacgtctcgtccgcatttgtgaggaccgtcacatagctgtctccggtcagcgtccatgcgctgtccgcatcgagggataccgccgccgcctgcgcggtgttctccgtgttgaccgcgcctgtaaaggtgctgccctgcgtaagtgaaagcgcaaaggtgctgatctcgtcgcacgtcacgtcgccttcaaaaacctggtttatacccgtcagggtaaactgcccgccatttgagccgggcgtgccccagttattggtctcgttgcccgccacattcgcaaggacgccgcttgtgaagacgagttccgtattttcaaagaacgcctccgcctgcgtattggtcacatagatcatcgggccgtccgacgtcgtcgtgagcctgctgtccgccgcttcaaagcgtgcggtcccctctgccgcgtcgccggaggtgctctgatagagcatgataccgttttcccccgccccggtgaggtccgtatccgtcatggtgatggagttctttccttccaccactgccgcctggctgcctgccgccgttcccgtgaggctatttaccgtgatgtcccccgtcgaatagatgcatgggctgccgtcgcccgccgcgaagaccgttccggtgtttaccgtgatggttccttcgccgcggtctgtagcgatgggcgcacaatgcgcaccttgcgtcgtaatatctacatttgtggcgttgaccgttccgccataggtggcgtcaaggccgcgggaggaattcccggtcgtatggatggtgacgttatccgccgtgaccgtggagtcctcacccgttgcaaagatggcgttcgagccttccgccgaggaagtcaataccgtatccgaaatctcggctgtgctgccgcctgccgccgcgaatacggcgtttaccgcataaaaattactttcatccgtactcgacgtatcgcccgttttatcgagcgtcccattttcgaggatcaggctgccgccgttttttacgagtacggtattttcgtctgcagacgaggaagcaatttcttcgccgtccgaggtcccgctggtgccgtccaccgtaaatacgcccgcaagctcgatacttcccgtctccgagccgctcaaatccccgccggggccgcccgcgccgccggcatttataatctcaagggtcgtgggaacaaaggattcgctttcgcccgatccttccatcttaacaaccgtaccttccgtaaggccggcgggcgacgcctctgtctcgctgccgccgtcctgttttgtgatggtgaccgcagtaaggtcgacggtgatttccgttccgtcttcatccgcggtaaagccgcccgccccgccggggccgccttcgcccagatcaggtggtgtgccgccgtttcctgcgtcttcggacggcgcttcaccgcttggcatatccggcgcctcccccgacggcatttctccgctttccagatcgggcggtgtgccgccactcattccgcctgtcaccgtcccgagggcaatcgtcgcttcattcccgcttaccgcggttaccttcccatatatcgcgtcatcctgaacctgcgcgcccgtttcctccgccggggcagcggacgaagtctccgccgcctgctgcgcacaggcggtaaaccccatcgccagaaccgcgcacagcaggaccgttaatatttttttcataccagttgttgctcctttcttggtctggtcccattatggaagcgtaacgtgaaggctgtgtggtgaaaggatgaaaaatctgtaaacgccaaaaaacgcccgcggggaaattccccgtgggcgttttgcgcataatttaccattcttccgtatacatatggatcggataggagaggtattccagcggatcaagctggtcgaccgtcacatatcctgccggtgcgttcaggattgtcggaatacggttcacaacggtcgcgcaggtgtgctccactgtcgctggtttgaccacgtgaaactccgtatcaggttcgccgacaatcttccagtcgcacatatcgccgtcgtccggcccgtataccttgccaatacactgggtctcgatcaccgggccctggtgcgtctgcgtcgtgacgaccgcgctcattccgatacagttgcccttcgggatcgtctccccggtggtttcagaatacaggtctttaccgtaaaaatacggtacgctcttttgcgtctgggatttgatcgtccagcccattttggcgcacagcgcctcgttggaattccacacataactcggttcgagaacttccgggtgggccagttgcttttcaaattcttccggtgtgagtcctacgccgtgcgcctgcgcgagcgcgagtccgtaatcctccacgttatagctgaccgctccttcgatgcggtctatagtatggacgccgcccgcaacgcccgcgatcatgttcacccaatagatatcctgcatccccgagcccacaatagtgcagcccgtttccttggcgaggacgtccagcttattcgtaagggcgggcgaggtcgtccacggatagatcgcttcttcacatgtcgtcactacgttgatgccgcgcgcggcgcatttttcgatatgcggatacatatcgtccatgaagctgaacagtgtaagaaccgcaatatccgcatcacattcgtctaggacagcatccgcgtcactgcggatcggaacgccgaggcgcacgccaaggcctgcccattcccctacgtccatgccaacgatgtctggattcacatcgaccgcgcccacgatatccgcgcccttctcatacaaataccgaaccgtatactttgccatttttccgcagccatattgtacggcacgaattttttgctgatacatattttctccctcttttcaaaaatttccaccgatgtccgcttgtgttgttttccccctggatactatcaatataccacccataagacacctcaaacctttttttaatcaaaagcgcccggcgggatcgccgggcgctgcttttcactttagctggaactgcggatcgcttcaagcggactcatcttcgttgccttccatgcaggataaatgccggacacaatgccgacgcctacggcgatcgcaatcgcgccgaggctgagccaccacgggatggaaagctgtaccccgttctggaaatacatgccgaggaagctgatctcgctgccgccgaaatttgcggccgcgccgaagatatagctgaccccgaggccgatcagccctcctataaggccgatcattgccgcctccacgaggaacatcaggttgatcttgcgaatggaaaggccgataaccttcattacgccgatctcccgcgcccgttcaaggatgctcgcgagcatggtattggcgataccgatcgccgataccagcagggagatgaggccgatggcgatgagctgcgactgctggcgcgcctgttcctcctgcatctgggtgatccattcggtggggctgtaggcctggaatcccatttccttgaccgccttgaggatgtcttccaccgcgtcaacatccaccgcgttcacatagccctgcgaatattgtccttctttgattcctgcctggtccatcagcttgcggttttcgcggataagcatattggcggcgggaatactcatatagatattatagctctcctgactgtccgactgcttaagaatgcccgtaatttttccgcggtattccttgggcgccggctggtcaggggtggatgggtctggattttcgatcaggtatccgctgcccgggtaatacttcacgttcgtccctacccagtctacatccggcccggccggttcctggtattcgccgtcgtcggccacggcgaccgcgtagccgccagccatcatcacgccgcccatcatgcctccggaggcattcgggtcctggaaatactgccgggcgttcgcgcccacgataagctccacgttagggctgtccgagaagacatttccctccatgaatttaaaatccatagtttgcgggtcaaccgcataaatataggtatccgccttatattttcccatcacaatatacgaggggatgctcatcaggccgctcgcgtttttcacccccggaatggcccgtatctggttcagcttggcatccgtaagcttggcgttttcccccgagcccatgggggatatctcgatccgcgtaaggtccgtactctgcatgatgctctggttgaactgctcaaggttcccgagaccgagcgccaccatgataacaatgcacgccgttccgataatcacgccgaggacggtcagtatggtccgcatcttgcgccgccacagattggatgccgcaagaagaacaatatcaataaatgtcatcgtcttcgaggtcctccgaaagctcctgcgcgcgtttttttcttttcctttttaccacaattacaatggccacggctgccgcagcgcctgcggtgatcccccagccccaccacgggaaacctgctggctcctccggcgggagctcttcgccgggatccatgccgccataatccggcataggcgtgaggactgtttcaaccggcaccttcatctcgtattcttcgccgtactcgtcttcataagtgacgacaaaatttccttccaccggaccgggctggacggcatcttccgcgcctgcgccgcctacagcctgcaccgtgccgctttcccccgatacaacaatcgagtccggtccatccgcaccgccggttattgcgccgtccgccgcgggagccacttcggcattcccggtttccgcgccgctcatgccgcccatccccgcattcgggtcctgggcaacggacgcatagatatccgccgtcttcgccgtgccggattccatatttccaagaaaagcgctcgattccggcgtaagaccgggaacgtcaagcacgaccgttacattataaagggtgttttttcccttgttgtataaattgaggctcgcggaggtggtatcgcccatatagacttccgatgggaatttcggcggatcatattcaagacggatcttctggtgcacctccacgctgatctcatccgccgccgtaagctgcgccgcctggctgtcttcgtaggcgatggcgatatcgatcttctgcggccccgctttggcgtcggcgcgcgcttccatcgtgaagctgaaatccgccgttttcttcgggccgatcttttcaatatacgccgtattggtattgtcgccgggcatcagatcggtcgtctggcttttgaaggtgactgtgatgtttttgacgctcgtcgatttgctcgtgttggtaagcgtcacctttacggtaaacttttcacccgcgttgaccggatcgggactgatctcgtaattgctgatgatgaccttgggctgagaactcactccgctccccccgccaccgccgcccgcgccgggatcgatccccgggtctgttcccgggtctgttcccgggtcttccgtcggctgaccggacggctccccacttggttcggccgtcggctcagaggggtcttgcccgtccgttaccttgacgaataccgtaaactcctgcgttttaacagtgccgtccataagcgggtatttcgcggtgacgacaaccggatagttcccattcaccctgttttccgcaagcggcaggctgaggtccaccaaataagactcctttgttttcccgtcggccagcttattttccttaagggatacggccgtatcgtaattattgaatataaagggagcggtcttgggatcgccgaggccaaccgaaatattgacctgttccccatttttcacctgttctgttcccgggccggggaccagcggaaggagaatgcgcgccgttcccccggatacggcggggctatatccctcctgataggaggcgttcatgcccccgtattgacttttgccgtcgatccggagcgccgttccgcgcatatccgcagcaaatgcggttggcagcgcaaacgaaaacgcgagtataacgatgagtgcgataattgtgatccgtttcttcatttttcctctccctgtattcctaattccgcctgtatgatctcttcggtacttatttcggcaggtccgtgcgtttcgcgtttcgccactttcccgtctatgatatgaataacctggtcggcatattccgccttggctggatcgtgcgtcacgaacaggagggtcgcgccgcgtttttttgcctgtccgcacataatgtccataatctgttccgcggtcaccgaatcgaggttgcccgtcggttcgtccgcaaagataatctccggctttgcgataatggcgcgcgcgatcgatacgcgttgctgctggccgcctgacagctggtttggaagatttttaagctgctgcctgaggcccatttccacaagcagtttttttgccgcttccatgcgcctggcgcgcggcacgccccggaacatcaagggcagcgccacgttttccaccgccgtcatggtgggcatcaggttgaacgattggaagacgaagccgatatgcttcaaacggaaatccaccaattcgctttcattcattttataagtcggttttttcttgataaaaatcctgcccgcggtcggatgttcaagccctgcaagcagggaaagcagcgtcgattttccgctgcccgaggtgccgagaatcgcgcacacctctccgggcatcacatcaatgtccacaccgtccagtgcatgtatttttgcactgcccatgcggtatatttttttcagattcctggctgcaatgatcggttccaaagacttcctccaaaaatggcgtctttcgggcataaaaaagccctgcacgaagtgaaggggaaacggcaaagatcccacccctaacctacgtctttcattatagcataaagaatctttccgttgggcggcactttatgaataaattcttacaaataatatggttttttaaaaattattatcgggacggcggaaggccgcgaaccgtcctccgcagaatatagagaagggaaacgcggatgcgtttcccttctctaattttatagtcctgcggtttccgttctaagtatacggcaggctcggttctcaagcgaacggcgctgtttcagccctcgatgatcttgacgaacgattggtactgcgcagcccattccgccgtatcccggggggtatacgtcttcggcgcacaggacgcggccaccacgcggcgcgcctctttgatgtccttgatttcacccgcggcaatgagctgtactacgatgttaccaagcccggttgcctcgtccggtccggcaattacggtcatgcctgtcgcattggccgtcatctggcacagaagcgcgctctttgtgccgcccccaaggatatgcaccgtaccgtattttttgcccgtgcattccgcgaggcgtttgatgctgtaatgaaacttcatggccaggctttcgtcgatgcagcggatgatttcgccttccgtctgcggaacaggctgccccgtcttttcgcagtaggcgcggatacgcctgggcaggtttccaggctttccaaattccgggctgtccacatcgatcaggctacggaacggcgtgcattctttcgccatctgctccagctctccaaagccgaactccttgccctcgcggatccactggcggcgggactcctggatcggccacgtacccattatatttttcaggaaacggtaagtgccttccaccccgccctcgttcgcgaagttgtatcccgctgccttttcgttgatgcagggttcgtgcagctcggtgccaagcaggctccatgtaccgcagctgatgaaaataaaatcatcttcctgcgtgggggtcgccgcgaccgccgccgcagtgtcgtggccggcagtacacatcactttggctttgggggcgccaagttcttcgcagatgtcgtcgcgcagcaggccgcactcggttcccggctggacgatatcacataatttttcgcgcagaatgcccgaaagctccagcaccttttcactccagtccctgcttttcgcatccagcatttcagatgtggacgcaatggtgtattccgtatgcatttcccccgtaaggaaatagttgtaaagatccggcgtaaagagtatttttttcgtcgccgccagaagctcgggcttctgctccttcaggtaaagcagttggtaaagcgtgttgatggctagatgctgcaggcccgccgtctgatacagctcgtccggatcaatccgcttcatgaattcctcaaacggctgttccgaccgcccgtcgcggtaatgaacagggttctggacaagctggccgtcttccccgatcaagccaaaatcgacgccccatgtgtcgatgccgatcatatcgaagcctcccgcaagcgccgccttgctgatgccctgcttgatattgtaaaacaggcgcagcgcatcccagaaaagcgtcccgttcagcgaaaccggatcattggtgaagcggtggagttcttccattgtaatttttccatccgcatactgggcgatcattgcacggccgctggaagcgccgaaatcgaatgctaaaattctgctcatgctctttcccctttactatttctattatattaactatactgaaaataagaatcggaatcaatgaagaaaacaacaaatttttgtaaattttcaacactctttgtttggatatgcggtttttttgtttttcccggaattgatgtcacgtttcgtttgtgagataataaaaatataatcatcacccagcctgagaggacggaggaaagatcatggactatgtaaagctcggcagaacggatctgaagataagccggatggtaatgggcggagacgtgcgcacaccccgcgaggaatttgccgccgcgctgcagcttgcggtggatcatggcgtgaacctcatcgataccgcgcccgcctacgggaacagcgaggaggtccttggcacgattctcccggagatcaggggcgagttgtttgtatctactaaattcagcaacgacccgcagccttttttgccgcaggaccgggactgcctgatgcgtgccgtggaaaacagcctctcccgcttgaaacgcgataagatcgatatctggatggtgcatgagcccgaccgtccgtatcaatacgactggtgggaacgcccgcaggaccttaatcatcctgaagcgtattatgggccggtcatggatgtgctgtccgacctgaaaagccaaggcgttatcaaatatacgggcatcggcgggacgacggcatatgagatcgtgccgatcatcgaaagcggctttttcgatgtgatactgactgccttcaactacagccttttgtggcaggaggcggccatcgaggtaatccccgcggcgaaaaagcagaatatggggattttgcttggctccgcgctccagcagggcgcgctcgcgaagcgctacgacgaacaggtaagggcaaagccggcatggctttcaaagccccgccagcagcaatacctgaaattatacgagctgtgcgacgatttgaagatcgatatcgcagaccttgcgatgcggtttgcattttcaaatccggatatccacgccgtcgtaacgggcgcgggcaaggtgaagaatgtgcagcggaaccttgagtcgtaccacaggggcccgctgccgcaggatgtgctcaggcgtatcggtgaaatttaccggatggtacccttccgcccattcgaggaatcgtttttcatgccctttacgcgcccctataaagggccgcgggcggtgcactgaacggcttcggctttccgcacatccgccaaataatttttgtattaaaaaaggacaggcaaccgcccgtccttttttgatcccggtttatttcttcttcagctcgtcgtccgtaatctgcaggctcctgcgtacgatcttcttggaaaggaacttgtccgcgcgtttttccacaatgcgttcaaagccccgcatcatgaacggcgcgaccgccgtgaaaacacacactacaacggcctgcggaaggctgattggcgccacatcgaaaaagcccctgaggaaaatgatgcacaggatgaacaggccaatggcgctgccgaggatcaccttgcgcgccgtggtaaacggcatgctgaccttagctaccacccacataccgaccgcaaccgccatgagcatgcacatcgtggagaccgccgccggggtgtattggaagattcccgcaagcacgttgaccgcaaggattccgataataacggaaagccctccgggcaatgatttttttaaaatattaatcaggaaatgctccttgatcggcgcaaaattcggctccaaagcaaggaaaaacgccgggaagccaatggtcacaaaactgattagcgtcatctgcaggggcgtaaacggataactgctgtcgagcaggattaccgtcgcgaacgcaaggagcaccgcatacaccgttttggtaataaacagagtggccacgcgttctatgttgttgattaccttgcgcccttcatttacaatatcgggcatcgtcgcaaaattagaatcgagcagcacaaggttggcgatatttttagccgcgtcgctgcccgacgccatagcaatgctgcaatcggattccttcagggccagcacgtcgtttacgccgtcgcccgccatggcgaccgtatgacccttgcgtttcagcgcggcaatcaattcctttttctgggaaggcgttacccgcccaaacacgctgtacttttcggcagccgcctcaatatcctccttcgttttgagcagggtggcatccaccgcttcataattttttacgcccgcgcgtttggcgatcgcgccggccgtgaccggactgtccccggatatgatcttaagctctacctgctgcttctcaaaaaattcaagcgtctttttcgcatccgcccgaatccggtcggaaatgagcaggaacgcgatggggcgcattcccccggggaccttgttatccagcacgtcggtggcgtcgctgtgcgcaagcaccagcacccggcttccttgtttcgcgtatttttccgcgtcgaggcccgcatacccgttgggaaaaataaactcgaaggccccgaggaaataggtcccttccccttcaaagcttacgccgctgtatttacgcgccgaagaaaatggcacgatggtttttacttcatggtcaaagctcttgccgaatttttccactacggcatggaaagtcgcgttatcgtcctgaagcgctccggccatattggagaggatcctgtccaagtcttttgcagcgcctacgggaatgacttcctccacatgcatctttccctcggtgatcgttcccgtcttgtcaaggcacaggatatctacacgcgcgagcgtctcgatgcagtagaggtcctgcacaagcgtctgctttgtcgcgaggatcacggcgctggttgcaagggaaatactggtgagcaggataaggccttcagggatcatgccaataagggcccccaccgttttgaccgtattatccggtatgggcaggcccaccacaagcccctgtttcagaaacagcagaatacccaccggcacgatgatgatgctgataattttaaggatgcggttgagggaattgcgcagctcggacgtacgctttttagatgccttcgcatcataggaaattttatttgcatagttttcaattcccacgcgctccacacgcgcgtaagcgttgcccgatacgataaagctgcccgaataaagaaagtcgcccgggcccttggccacaacgtcggattcgcccgtaatcagggactcgttgacctccaccgatccttcaagcatcaccccgtccgcacatacctggttgccgggccgaagctgcatcacatcgtcaaggacgatatccgcaacgccgatctccttttcggctccgttccgtaccaccgtgaccttggccgccacgatcaccgagagcttatcgagcgttttcttggttacgatctcctgtgcaatcccaatgacaaggttccccacgacaacgagcaggaaaacaagttccttgatgtagcccgtcatcacgataaggaccgtgaacaccatattcagtatattaaaaaaggttacaaggttatccttaaaaatctgccggtacgattttgtcgtattcgtattatacgcatttgtgagacccgcatcaatacggcttttgacctgtccgtccgtaagccccgtgcggcagtccgaatcatagcgctgttgcccgtcgtccatgtcgttccttccccgtcattacatagtgactttattatatcatatttaccctatgctccaatgattatccgccaaaatttataaattcctaacaaaaacgacggatttcttcatccgccgtttcaaaagtcttaagaattcccttatttcgtgaggaaccggtattccgttacgtcgtggtaggtctttcccttctgcaggatgcaggaaggaaattcatcgtggtgcacgctgtccgggaaaagctgcgtctcaaagcaataggccgtacgcgggccgtattgcactccgttcttccccttcatctccggcacgaagttcgccgtatagagctgcatgcccggtttgtccgtatagacctccatcacgcggccgctctgcgggtcttcaacccgcgccgcacgccggaagcccgcgccccacaaaacaaagttgtggtcgtatcccccgcacagcttcatctgcgggaatggatcttcgatccgctcgccgatcaggtgcggatgcgtgaaatcgagcggagtgcccgcaacctcggcaatctctcccgtggggatcaggttgccgtccaccggcgtatattccccggcgtcgatggtcagcacatggtcgctgatctcgccctccccccgcaggttgaaataagaatgatttgtgagggacaatatggtgtcctcatccgattctgcaaaatattcgaggcgcactgcattatcgtccgtaagggtcatttcaatgcgcacatccaccgcgcccgggaagccgccttccccgtccggactgtgataggaaagggtcagcatattgccgtctgccgtaccgtcccacaccttgcggttaaagctgttgtctcctccatgaagaagatttaccccgttttcgttgcacgggatggcgtaatcctttccgcccaccgtgaaccggccgtttgcgatccggttggcataccgcccgactgttgcgccaaaaaagggatggccgccaaaatactcctccggcgtttcaaagccgagcactacatccgccctattgccgtctgcgtccggcatttcgaccgacataatcgtcccgccataattcgtgaccttcatgcatccgccggcttcactcgaaaatgtataaacaaaaatatccttaccgtctttactgccagttttttgagtttcaatcatgatctttaccgcccttttgatttgctattattattctacatcaaaagcggcttttttcctatccgttatcaatcaattttttgtgaaattttcttttgcaatatgaaggaggtgtgctataatagttagcagtggaggcatagctcagttggtcagagtacctgcttcacatgtaggg

SEQ ID NO: 2 - DNA, 978 bp, Christensenella minuta
  misc_feature: CP029256.11933575-1934552 BSH gene
atgtgtacagcaataacgtattatacaaaggatcactattttggaagaaatctggatctggaattttcttataatgaaacagtaacggttacaccaaaatattatccgtttcattttcgtaatggaaaagtgctcaatcatcattatgcaatgatagggatggcttatatagttgatgactttccgctttactacgatgcaacaaatgaaaagggtctcagtatggcaggactgaacttcccggataatgcggactataaagaagtgaaggaaggatacgataatattgctccctttgaatttattccatggatactcggtcaatgtgcaagtgtgagtgaagcaagaattctcttagaacagattaacttggtaaatttgaactttagtgaagagctgccgctttctccattacattggatgatttctgaccagcgagattccattgtagtagaatcaacaaaagatggtttgaaagttttcgaaaaccctgtaggagttctgacaaataatcctacctttgactatcagatgtttaatctcaataattatatgcatttatcgaaagaaccccctgcaaatacatttgctgccgaattggaacttgaacagtatagccggggtatgggggcgataggccttcccggagatttatcatccgcttcgagattcgtgaaagcagcgtttacaaagatgaactcggtttccggtgattcggaatcggaaagtatcagccagttttttcacattcttggctcggtagagcagcaacgaggctgcgtacatcttggtgaagataaatatgaaataacaatttattcttcctgctgtaacatggacaagggaatttattactatacaacttatgaaaacaaccagatcacagcagttgatatgtataaagaaaatctggacggaaacactataataagctatccgcttatgaaagaacagcagattaactatcggaattactaa

SEQ ID NO: 3 - DNA, 129 bp, Christensenella minuta
ctcggtcaatgtgcaagtgtgagtgaagcaagaattctcttagaacagattaacttggtaaatttgaactttagtgaagagctgccgctttctccattacattggatgatttctgaccagcgagattcc

SEQ ID NO: 4 - DNA, 121 bp, Christensenella minuta
aagaaccccctgcaaatacatttgctgccgaattggaacttgaacagtatagccggggtatgggggcgataggccttcccggagatttatcatccgcttcgagattcgtgaaagcagcgtt

SEQ ID NO: 5 - DNA, 86 bp, Christensenella minuta
tgctccctttgaatttattccatggatactcggtcaatgtgcaagtgtgagtgaagcaagaattctcttagaacagattaacttgg

SEQ ID NO: 6 - DNA, 20 bp, Christensenella minuta
  misc_feature: BSH_qPCR_F2
ctcggtcaatgtgcaagtgt

SEQ ID NO: 7 - DNA, 20 bp, Christensenella minuta
  misc_feature: BSH_qPCR_R2
ggaatctcgctggtcagaaa

SEQ ID NO: 8 - DNA, 20 bp, Christensenella minuta
  misc_feature: BSH_qPCR_F3
aagaaccccctgcaaataca

SEQ ID NO: 9 - DNA, 20 bp, Christensenella minuta
  misc_feature: BSH_qPCR_R3
aacgctgctttcacgaatct

SEQ ID NO: 10 - DNA, 21 bp, Christensenella minuta
  misc_feature: BSH_qPCR_F6
tgctccctttgaatttattcc

SEQ ID NO: 11 - DNA, 23 bp, Christensenella minuta
  misc_feature: BSH_qPCR_R6
ccaagttaatctgttctaagaga

SEQ ID NO: 12 - DNA, 26 bp, Christensenella minuta
  misc_feature: BSH_qPCR_probe2
agtgaagagctgccgctttctccatt

SEQ ID NO: 13 - DNA, 27 bp, Christensenella minuta
  misc_feature: BSH_qPCR_probe3
actgttcaagttccaattcggcagcaa

SEQ ID NO: 14 - DNA, 27 bp, Christensenella minuta
  misc_feature: BSH_qPCR_probe6
cacacttgcacattgaccgagtatcca